(19) United States
(12) Patent Application Publication
(10) Pub. No.: US 2019/0271022 A1
(43) Pub. Date: Sep. 5, 2019

(54) Signal Polypeptide For Improved Secretion Of Protein

(71) Applicant: MedImmune, LLC, Gaithersburg, MD (US)

(72) Inventors: Soojin Han, Gaithersburg, MD (US); Ronald Schoner, Gaithersburg, MD (US)

(73) Assignee: MedImmune, LLC, Gaithersburg, MD (US)

(21) Appl. No.: 16/344,993

(22) PCT Filed: Oct. 26, 2017
(86) PCT No.: PCT/US2017/058413

§ 371 Date: Apr. 25, 2019

Related U.S. Application Data
(60) Provisional application No. 62/413,607, filed on Oct. 27, 2016.

(51) Int. Cl. C12P 21/02; C07K 14/435; C12N 15/62; C12N 15/70

(52) U.S. Cl. CPC C12P 21/02; C12N 15/70; C12N 15/625; C07K 14/435

(57) ABSTRACT

The invention relates to a signal polypeptide for improving excretory production of a heterologous polypeptide, proteins comprising the signal polypeptide, nucleic acids encoding the signal polypeptide, and methods of using thereof.

### SEQUENCE LISTING

The instant application contains a Sequence Listing which has been submitted electronically in ASCII format and is hereby incorporated by reference in its entirety. Said ASCII copy, created on Oct. 19, 2017, is named NSP4-100-WO-PCT-SequenceListing and is 17,021 bytes in size.

### FIELD OF THE INVENTION

The invention relates to a signal polypeptide for improving excretory production of a heterologous polypeptide, proteins comprising the signal polypeptide, nucleic acids encoding the signal polypeptide, and methods of producing thereof. Exemplary embodiments include an improved signal polypeptide attached to alpha toxin, and methods to produce and isolate alpha toxin.

### BACKGROUND OF THE INVENTION

Expression in bacteria can be the method of choice for the commercial production of pharmaceutical and industrial proteins. The secretion of commercially important heterologous recombinant proteins into the periplasm or culture medium in E. coli offers several advantages in the production of commercially important recombinant proteins, including cost and time savings, reductions in endotoxins, growth on inexpensive carbon sources, rapid biomass accumulation, amenability to high cell-density fermentations and simple process scale-up. (Mergulhao et al., Biotech Advances 23:177-202 (2005); Gottesman et al., Annu. Rev. Genet 30:465-506 (1996)). For these reasons, many studies such as signal peptide modification and coexpression of chaperone have been conducted to improve the secretion efficiency of recombinant proteins in E. coli expression systems. (Baneyx at al., Nature Biotech 22:1399-1408 (2004); Choi et al., Appl. Microbiol Biotechnol 64:625-635 (2004); Cornelis et al., Curr Opinion in Biotech 11:450-454 (2000); Klatt et al., Microbiol Cell Factories 11:97-106 (2012)).

In E. coli secretion pathways, signal polypeptides play a critical role in translocation and secretion of recombinant proteins during the secretion process. A number of signal polypeptides have been studied to improve secretion of recombinant proteins in E. coli system. (Humphreys et al., Protein Expression and Purification 20:252-264 (2000); Velaithan et al., Ann Microbiol 64:543-550 (2014); Jonet et al., J Mol Microbiol Biotechnol 22:48-58 (2012); Ismail et al., Biotech letters 33:999-1005 (2011); Low et al., Bioengineered 3:334-338 (2011); Nagano et al., Biochem Biophys Res Commun 447:655-659 (2014)). In general, recombinant proteins are secreted into the periplasmic space through the cytoplasmic membrane and signal polypeptide is cleaved by signal peptidase during the export. Usually, the secreted recombinant proteins are extracellularly released into culture medium through the outer membrane from the periplasmic space.

Signal polypeptides are located in the n-terminal region of recombinant protein precursor, which is recognized by a signal recognition particle (SPR) when nascent polypeptide chains emerge from the ribosome in the secretion pathways. Signal polypeptides have a length of 20-30 amino acid residues and three distinguishable structural features. These three regions are composed with an amino-terminal region with a net positive charge (the n-region), followed by a hydrophobic region (the h-region), and then a protease recognition sequence (the c-region) with a preference for small residues at the −3(P3) and −1(P1) positions relative to the cleavage site. (Mergulhao et al., Biotechnology Adv 23:177-202 (2005); Paetzel et al., Biochmica et Biophysica Acta 1843:1497-1508 (2014); Paetzel et al., Pharmacology & Therapeutics 87:27-49 (2000)). Especially, the h-region is the important region to protein translocation across the bacterial cytoplasmic membrane during the secretion process because translocation efficiency increases with the length and a hydrophobicity of the h-region, and a minimum hydrophobicity is required for function (Wang et al., J. Biol. Chem 275:10154-10159 (2000)). A number of published literatures have been shown that modification of signal polypeptides improved the secretion of a heterologous protein in E. coli expression system (Humphreys et al., Protein Expression and Purification 20:252-264 (2000); Velaithan et al., Ann Microbiol 64:543-550 (2014); Jonet et al., J Mol Microbiol Biotechnol 22:48-58 (2012); Ismail et al., Biotech letters 33:999-1005 (2011); Low et al., Bioengineered 3:334-338 (2011); Nagano et al., Biochem Biophys Res Commun 447:655-659 (2014)).

### SUMMARY OF THE INVENTION

The current disclosure is directed to a signal polypeptide comprising an amino acid sequence having at least 90% sequence identity to SEQ ID NO: 1. In some embodiments, the signal polypeptide comprises an amino acid sequence having at least 95% sequence identity to SEQ ID NO: 1. In some embodiments, the signal polypeptide comprises an amino acid sequence of SEQ ID NO: 1.

The disclosure is also directed to a protein comprising (i) a signal polypeptide comprising an amino acid sequence having at least 90% sequence identity to SEQ ID NO: 1, and (ii) a heterologous polypeptide. In some embodiments, the protein comprises (i) a signal polypeptide comprising an amino acid sequence having at least 95% sequence identity to SEQ ID NO: 1, and (ii) a heterologous polypeptide. In some embodiments, the protein comprises (i) a signal polypeptide comprising an amino acid sequence of SEQ ID NO: 1, and (ii) a heterologous polypeptide.

Various heterologous polypeptides can be used in the present invention. In some embodiments, the heterologous polypeptide comprises greater than 20 amino acids. In some embodiments, the heterologous polypeptide has a molecular weight of 25 kDa to 50 kDa. In some embodiments, the heterologous polypeptide has a molecular weight of 30 kDa to 35 kDa. In some embodiments, the heterologous polypeptide is selected from the group consisting of an enzyme, toxin, antibody, antibody fragment, antigen, therapeutic protein, and combination thereof. In some embodiments, the heterologous polypeptide comprises Alpha Toxin (AT). In some embodiments, the heterologous polypeptide comprises Alpha Toxin from Staphylococcus aureus. In some embodiments, the Alpha Toxin comprises a substitution at the amino add position corresponding to H35. In some embodiments, the substitution is a H35L substitution.

In some embodiments, the disclosure is directed to a protein comprising the amino acid sequence of SEQ ID NO: 2. In some embodiments, the disclosure is directed to a composition comprising a signal polypeptide or a protein as described herein.

In some embodiments, the disclosure is directed to a nucleic acid encoding the signal polypeptide as described herein, e.g., SEQ ID NO: 1, or a protein comprising the signal polypeptide as described herein. In some embodiments, the nucleic acid (1) encodes the signal polypeptide as described herein, and (2) comprises one or more restriction enzyme sites.

In some embodiments, the disclosure is directed to a vector comprising a nucleic acid as described herein. In some embodiments, the vector further comprises an origin of replication. In some embodiments, the vector further comprises a promoter sequence operably linked to the nucleic acid. In some embodiments, the promoter sequence is operable in a prokaryote. In some embodiments, the vector is a plasmid, a transposon, or a viral vector.

In some embodiments, the disclosure is directed to a recombinant cell engineered to express a protein described herein comprising the signal polypeptide. In some embodiments, the recombinant cell is a prokaryote cell. In some embodiments, the prokaryote cell is of the genus Escherichia. In some embodiments, the prokaryote cell is Escherichia coli.

In some embodiments, the disclosure is directed to a host cell transformed with a vector as described herein. In some embodiments, the host cell is a prokaryote cell. In some embodiments, the prokaryote cell is of the genus Escherichia. In some embodiments, the prokaryote cell is Escherichia coli.

In some embodiments, the disclosure is directed to a method of producing a protein as described herein, comprising culturing a recombinant cell engineered to express the protein, or a host cell transformed with a vector encoding the protein, under conditions in which the protein is expressed. In some embodiments, the method of the present invention comprises recovering the protein from the cell culture. In some embodiments, the recovering the protein comprises centrifugation to remove cells and/or cellular debris. In some embodiments, recovering the protein comprises filtering to remove cells and/or cellular debris.

In some embodiments, the recombinant cell or host cell is cultured in cell culture under conditions in which the protein is secreted from the recombinant cell or host cell. In some embodiments, the recombinant cell or host cell is cultured in cell culture under conditions in which the signal polypeptide is cleaved from the protein.

In some embodiments, the disclosure is directed to a method of increasing the rate of protein secretion from a cell, comprising: (a) culturing in cell culture a host cell comprising the nucleic acid or the vector as described herein which encodes the protein, (b) inducing expression of the protein, and (c) recovering the protein secreted into the supernatant of the cell culture, wherein the rate of protein secretion is compared to the rate of protein secretion of the protein with a dsbA signal polypeptide. In some embodiments, the recovering of (c) occurs between 8 and 12 hours after the inducing of (b). In some embodiments, the rate of protein secretion is increased greater than 20% per hour.

In some embodiments, the disclosure is directed to a method of increasing the quantity of a protein secreted from a cell, comprising: (a) culturing in cell culture a host cell comprising a nucleic acid or a vector which encodes the protein as described herein, (b) inducing expression of the protein, and (c) recovering the protein secreted into the supernatant of the cell culture, wherein the quantity of protein secreted is compared to a protein with a dsbA signal polypeptide. In some embodiments, the quantity of the protein secreted from the cell is increased greater than 20% compared to a protein with a dsbA signal polypeptide. In some embodiments, the quantity of the protein secreted from the cell is increased greater than 100% compared to a protein with a dsbA signal polypeptide.

In some embodiments, the disclosure is directed to a method of making a protein, said method comprising a) culturing a host cell comprising a nucleic acid or the a vector as described herein, so that the nucleic acid is expressed, whereby upon expression of the nucleic acid or vector in the host cell, a protein encoded by the nucleic acid or vector is secreted from the cell into the supernatant; and b) isolating the secreted protein from the supernatant. In some embodiments, the host cell or recombinant cell is Escherichia coli. In some embodiments, the host cell is cultured in cell culture under conditions in which the signal polypeptide is cleaved from the protein. In some embodiments, isolating the secreted protein comprises centrifugation to remove cells and/or cellular debris. In some embodiments, isolating the secreted protein comprises filtering to remove cells and/or cellular debris.

In some embodiments, the disclosure is directed to a protein made by any of the methods described herein.

### BRIEF SUMMARY OF THE FIGURES

FIG. 1: Screening bacterial signal polypeptides conjugated to AT_{H35L} in Micro24 bioreactor fed-batch process. Samples were harvested and spun down at 14 hours post-induction with 0.5 mM IPTG. Supernatants were loaded on a SDS-PAGE denaturing gel and quantified by Western blot using monoclonal antibody against AT_{H35L}. Arrow indicates the secreted AT_{H35L} protein in culture medium and purified AT_{H35L} was loaded as a reference.

FIG. 2. The schematic representation of novel signal polypeptide constructions used in this study. The figure illustrates different structural combination of novel signal polypeptides conjugated to AT_{H35L}. Used signal polypeptides conjugated to AT_{H35L} are described on the left of each construct. (FIG. 2A) Set I novel signal polypeptides, NSP1 thru NSP6, were created by modification of dsbAss and pelBss. (FIG. 2B) Set II novel signal polypeptides, NSP4a thru NSP4c, were created by amino acids shuffling or replacement of amino acids with Leucine or Alanine in the h-region of NSP4. (FIG. 2C) Set III novel signal polypeptides, NSP3a thru NSP3d, was created by amino acids shuffling in the h-region of NSP3. Numbers next to each amino acid in the h-region indicate the position of amino acid in the h-region. (Figs. A, B and C). D-N, D-H and D-C represent the n-region, the h-region and the c-region of dsbAss, respectively. P-N, P-H and P-C represent the n-region, the h-region and the c-region of pelBss, respectively.

FIG. 3. Secretion efficiency of AT_{H35L} by set I novel signal polypeptides in 1L fed-batch culture. FIGS. 3A and B. Evaluation of secreted AT_{H35L} in the culture medium by electrophoresis in denaturing condition. Arrows indicate secreted AT_{H35L} protein. FIG. 3C. Quantification of secreted AT_{H35L} in the culture medium by customized Octet assay. NSP2, NSP4 and NSP6 share the same h-region in their structure. Interestingly, secretion efficiency of AT_{H35L} was enhanced by NSP 2 and NSP4 whilst it was reduced by NSP6.

FIG. 4. Secretion efficiency of AT_{H35L} by set II novel signal polypeptides in 1L fed-batch culture. Evaluation and quantification of secreted AT_{H35L} were performed by electrophoresis in denaturing condition and customized Octet assay, respectively. Amino acids shuffling in the h-region affects secretion efficiency of AT_{H35L} (NSP4a). Although the h-region of NSP4b contains only strong hydrophobic amino acids as polyleucin, secretion efficiency of AT_{H35L} is reduced.

FIG. 5. Secretion efficiency of AT_{H35L} by amino acids shuffling in the h-region of set III novel signal polypeptides in 1L fed-batch culture. Altered hydrophobicity by amino acids shuffling significantly affects secretion efficiency of AT_{H35L}.

FIG. 6. Optimization of induction length for the productivity of extracellular AT_{H35L} in 1 L Fed-batch culture. FIGS. 6A and B. Asterisk indicates released AT_{H35L} pre-protein from dead cells due to overgrowth of cells. Arrows indicate properly secreted mature AT_{H35L} during the secretion process. FIG. 6C. Quantification of AT_{H35L} was measured by customized Octet assay.

FIG. 7. Codon optimized AT_{H35L} nucleotide sequences (SEQ ID NO:5) (FIG. 7A), AT amino acid sequence (SEQ ID NO: 3) (FIG. 7B), AT_{H35L} amino acid sequence (SEQ ID NO: 4) (FIG. 7C), and NSP4ss+AT_{H35L} (SEQ ID NO: 2) (FIG. 7D).

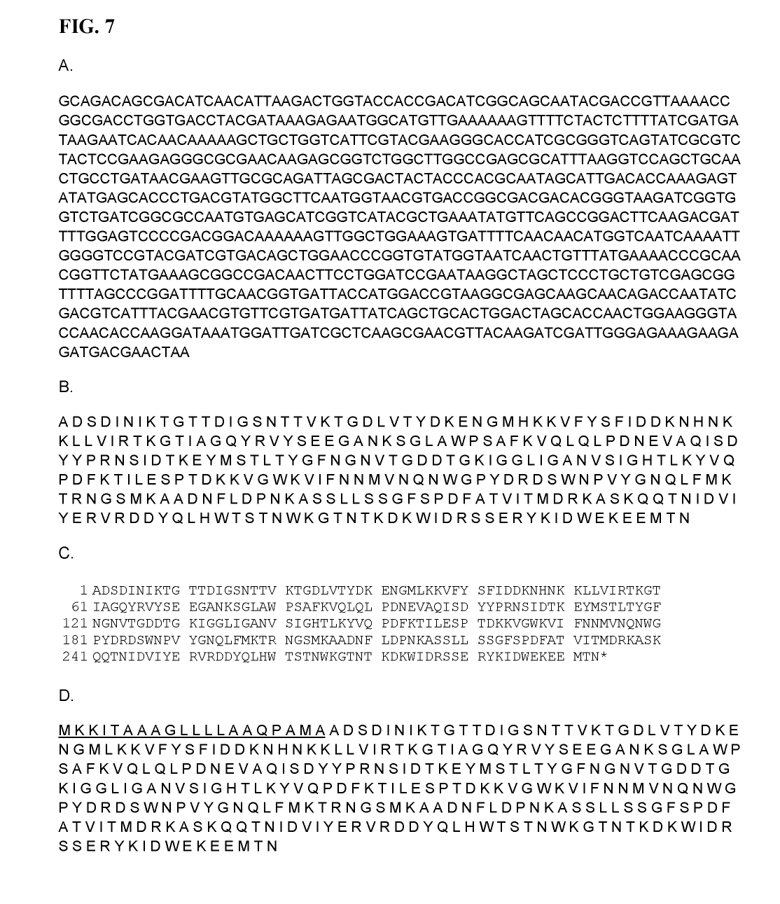

### DETAILED DESCRIPTION OF THE INVENTION

The present invention is directed to a signal polypeptide, and proteins comprising the signal polypeptide and a heterologous polypeptide, wherein the signal polypeptide provides for improved secretion of the heterologous polypeptide.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the method/device being employed to determine the value, or the variation that exists among the study subjects. Typically the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability depending on the situation.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or polypeptides, proteins, nucleic acids and vectors of the current disclosure, and vice versa. Furthermore, polypeptides, proteins, nucleic acids and vectors of the current disclosure can be used to achieve methods of the current disclosure.

### Translocation Systems

Novel signal polypeptides are provided which promote the targeting of an operably linked polypeptide of interest to the periplasm or into the extracellular environment. For the purposes of the present invention, the terms “signal polypeptide”, “secretion signal,” “secretion signal polypeptide,” “signal polypeptide,” or “leader sequence” are intended to include a peptide sequence that is useful for targeting an operably linked polypeptide of interest to the periplasm or into the extracellular space.

The signal polypeptide of the invention improves previous methods of production of recombinant proteins in bacteria. The signal polypeptide herein can increase the production (harvest) of proteins by increasing secretion of the protein from the intracellular environment. Secretion into the periplasmic space also has the well-known effect of facilitating proper disulfide bond formation (Manoil et al., Methods in Enzymol. 326:35-47(2000)). Other benefits of secretion of recombinant protein include more efficient isolation of the protein; proper folding and disulfide bond formation of the transgenic protein, leading to an increase in the percentage of the protein in active form; reduced formation of inclusion bodies and reduced toxicity to the host cell; and increased percentage of the recombinant protein in soluble form. The potential for excretion of the protein of interest into the culture medium can also potentially promote continuous, rather than batch culture for protein production.

In Gram-negative bacteria, a protein secreted from the cytoplasm can end up in the periplasmic space, attached to the outer membrane, or in the extracellular broth. The signal polypeptide herein can increase the production of proteins by increasing secretion of the protein from the intracellular environment into the periplasmic space, attached to the outer membrane, or into the extracellular broth. In some embodiments, the methods of the present invention can also reduce and/or eliminate inclusion bodies, which are made of aggregated proteins.

Prokaryotes, e.g., gram-positive and gram-negative bacteria, have evolved numerous systems for the active export of proteins across their membranes. Routes of secretion in gram-negative bacteria include, e.g.: the ABC (Type I) pathway, the Path/Fla (Type III) pathway, and the Path % Vir (Type IV) pathway for one-step translocation across both the plasma and outer membrane; the Sec (Type II), Tat, MscL, and Holins pathways for translocation across the plasma membrane; and the Sec-plus-fimbrial usher porin (FUP), Sec-plus-autotransporter (AT), Sec-plus-two partner secretion (TPS), Sec-plus-main terminal branch (MTB), and Tat-plus-MTB pathways for two-step translocation across the plasma and outer membranes. Not all bacteria have all of these secretion pathways. Thus, in some embodiments, the invention is directed to methods of increasing translocation of a protein across the membrane using the ABC (Type I), the Path/Fla (Type III), Path % Vir (Type IV); Sec (Type II), Tat, MscL, Holins, Sec-plus-fimbrial usher porin (FUP), Sec-plus-autotransporter (AT), Sec-plus-two partner secretion (TPS), Sec-plus-main terminal branch (MTB), and Tat-plus-MTB pathways using the signal polypeptide as described herein.

In some embodiments, the signal polypeptide as described herein utilizes the Sec secretion system. The Sec system is reported to be responsible for export of proteins with the N-terminal signal polypeptides across the cytoplasmic membranes (see, Agarraberes and Dice, Biochim Biophys Acta. 1513:1-24 (2001); Muller et al., Prog Nucleic Acid Res Mol. Biol. 66:107-157 (2001)), each of which is incorporated by reference herein. Protein complexes of the Sec family are found universally in prokaryotes and eukaryotes. The bacterial Sec system consists of transport proteins, a chaperone protein (SecB) or signal recognition particle (SRP) and signal peptidases (SPase I and SPase II). The Sec transport complex in E. coli consists of three integral inner membrane proteins, SecY, SecE and SecG, and the cytoplasmic ATPase, SecA. SecA recruits SecY/E/G complexes to form the active translocation channel. The chaperone protein SecB binds to the nascent polypeptide chain to prevent it from folding and targets it to SecA. The linear polypeptide chain is subsequently transported through the SecYEG channel and, following cleavage of the signal polypeptide, the protein is folded in the periplasm. Three auxiliary proteins (SecD, SecF and YajC) form a complex that is not essential for secretion but stimulates secretion up to ten-fold under many conditions, particularly at low temperatures.

Proteins that are transported into the periplasm, i.e. through a type II secretion system, can also be exported into the extracellular media in a further step. The mechanisms are generally through an autotransporter, a two partner secretion system, a main terminal branch system or a fimbrial usher porin. In some embodiments, the present invention is directed to methods of increasing translocation of a protein comprising the signal polypeptide described herein across the membrane using the type II secretion system.

Signal polypeptides interact with the proteins of the secretion systems so that the cell properly directs the protein to its appropriate destination. Five of the eight known signal-polypeptide-based secretion systems are those that involve the Sec system. These five are referred to as involved in Sec-dependent cytoplasmic membrane translocation and their signal polypeptides operative therein can be referred to as Sec dependent signals polypeptides. One of the issues in developing an appropriate secretion signal is to ensure that the signal is appropriately expressed and cleaved from the expressed protein.

Signal polypeptides for the Sec pathway generally consist of the following three domains: (i) a positively charged n-region, (ii) a hydrophobic h-region and (iii) an uncharged but polar c-region. The cleavage site for the signal peptidase is located in the c-region. However, the degree of signal polypeptide conservation and length, as well as the cleavage site position, can vary between different proteins.

A signature of Sec-dependent protein export is the presence of a short (about 30 amino acids), mainly hydrophobic amino-terminal signal polypeptide in the exported protein. The signal polypeptide aids protein export and is cleaved off by a periplasmic signal peptidase when the exported protein reaches the periplasm. A typical N-terminal Sec signal polypeptide contains an N-domain with at least one arginine or lysine residue, followed by a domain that contains a stretch of hydrophobic residues, and a C-domain containing the cleavage site for signal peptidases.

### Signal Polypeptides

In some embodiments of the present invention, a signal polypeptide is provided, wherein the signal polypeptide is a novel secretion signal polypeptide that can be used for targeting an operably linked protein or polypeptide of interest to the periplasm (of gram-negative bacteria) or into the extracellular space. In some embodiments, the signal polypeptide of the present invention comprises the DsbAss N-terminal domain and C-terminal domain, and the PelBss H-domain. In some embodiments, the signal polypeptide of the present invention comprises the amino acid sequence of SEQ ID NO: 1. In some embodiments, the signal polypeptide is an isolated polypeptide. In a preferred embodiment, the signal polypeptide is attached at the N-terminus of a heterologous polypeptide of interest.

The signal polypeptides of the present invention demonstrate the hydrophobic region (h-region) of the signal polypeptide and the position of the h-region amino acids significantly affect the secretion efficiency of proteins in E. coli. For example, as demonstrated in the Examples found herein, Alpha Toxin (AT_{H35L} protein) attached to the signal polypeptide of the present invention has increased secretion efficiency relative to Alpha Toxin comprising either the dsbAss or pelBss signal polypeptides. Additionally, the signal polypeptides of the present invention demonstrate that a shift in the position of the h-region amino acids without polymorphism altered the secretion efficiency of the protein.

In some embodiments, the signal polypeptide is MKKITAAAGLLLLAAQPAMA (SEQ ID NO: 1). In some embodiments, the signal polypeptide comprises an amino acid sequence about 70%, 75%, 80%, 85%, 86%, 87%, 88%, 89%, 90%, 91%, 92%, 93%, 94%, 95%, 96%, 97%, 98%, 99% or 100% identical to SEQ ID NO: 1. In some embodiments, the signal polypeptide comprises an amino acid sequence about 90% identical to SEQ ID NO: 1. In some embodiments, the signal polypeptide comprises an amino acid sequence about 95% identical to SEQ ID NO: 1.

In some embodiments, the signal polypeptide is substantially homologous or substantially similar to SEQ ID NO: 1. By “substantially homologous” or “substantially similar” is intended an amino acid that has at least about 60% or 65% sequence identity, about 70% or 75% sequence identity, about 80% or 85% sequence identity, about 90%, about 91%, about 92%, about 93%, about 94%, about 95%, about 96%, about 97%, about 98% or about 99% or greater sequence identity compared to a reference sequence using one of the alignment programs described herein using standard parameters.

In some embodiments, the signal polypeptide comprises a fragment of SEQ ID NO:1, which is truncated by 1, 2, 3, or 4 amino acids, preferably 1 or 2 amino acids, from the amino terminal or carboxy terminal, but retains biological activity, i.e., secretion signal activity. In some embodiments, the signal polypeptide comprises an internal amino acid deleted from the amino acid sequence of SEQ ID NO: 1, in which, 1, 2, 3, or 4 amino acids, preferably 1 or 2 amino acids, are deleted. In some embodiments, the signal polypeptide comprises an internal or external amino acid inserted into the amino acid sequence of SEQ ID NO: 1, in which, 1, 2, 3, or 4 amino acids are inserted.

In some embodiments, the signal polypeptide comprises a fragment of SEQ ID NO:1, wherein 1, 2, 3, or 4 amino acids, preferably 1 or 2 amino acids, are replaced, i.e., substituted, with a different amino acid. In some embodiments, the amino acid replaced is a conservative substitution, a highly conserved substitution, or a very highly conserved substitution. A “conservative amino acid substitution” is one in which the amino acid residue is replaced with an amino acid residue having a similar side chain. Examples of conservative, highly conserved and very highly conserved amino acid substitutions are found in Table 1.

TABLE 1

| | Very Highly | | |
|---|---|---|---|
| Original | Conserved | Highly Conserved | Conserved |
| Residue | Substitutions | Substitutions | Substitutions |
| Ala | Ser | Gly, Ser, Thr | Cys, Gly, Ser, Thr, |
| | | | Val |
| Arg | Lys | Gln, His, Lys | Asn, Gln, Glu, His, |
| Asn | Gln, His | Asp, Gln, His, Lys, | Arg, Asp, Gln, His, |
| | | Ser, Thr | Lys, Ser, Thr |
| Asp | Glu | Asn, Glu | Asn, Gln, Glu, Ser |
| Cys | Ser | Ser | Ala, Ser |
| Gln | Asn | Arg, Asn, Glu, His, | Arg, Asn, Asp, Glu, |
| | | Lys, Met | His, Lys, Met, Ser |
| Glu | Asp | Asp, Gln, Lys | Arg, Asn, Asp, Glu, |
| | | | His, Lys, Met, Ser |
| Gly | Pro | Ala | Ala, Ser |
| His | Asn, Gln | Arg, Asn, Gln, Tyr | Arg, Asn, Gln, Glu, |
| | | | Tyr |
| Ile | Leu, Val | Leu, Met, Val | Leu, Met, Phe, Val |
| Leu | Ile, Val | Arg, Asn, Gln, Glu | Ile, Met, Phe, Val |
| Lys | Arg, Gln, Glu | Arg, Asn, Gln, Glu | Arg, Asn, Gln, Glu, |
| | | | Ser |
| Met | Leu, Ile | Gln, Ile, Leu, Val | Gln, Ile, Leu, Phe, |
| | | | Val |
| Phe | Met, Leu, Tyr | Leu, Trp, Tyr | Ile, Leu, Met, Trp, |
| | | | Tyr |
| Ser | Thr | Ala, Asn, Thr | Ala, Asn, Asp, Gln, |
| | | | Glu, Gly, Lys, Thr |
| Thr | Ser | Ala, Asn, Ser | Ala, Asn, Ser, Val |
| Trp | Tyr | Phe, Tyr | Phe, Tyr |
| Tyr | Trp, Phe | His, Phe, Trp | His, Phe, Trp |
| Val | Ile, Leu | Ile, Leu, Met | Ala, Ile, Leu, Met, |
| | | | Thr |

In some embodiments, the deletions, substitutions or insertions maintain a similar hydrophobicity of the signal polypeptide. For example, in some embodiments, the signal polypeptide of SEQ ID NO: 1 has a similar (or in some instances identical) hydrophobicity in the H-domain of the signal polypeptide.

In some instances, 1, 2, 3, or 4 amino acids of SEQ ID NO: 1 change position with each other. In some embodiments, the deletions, substitutions or insertions of the signal polypeptide of SEQ ID NO: 1 retains the desired function of the original polypeptide, i.e., the signal polypeptide is capable of facilitating the secretion of the attached protein from the intracellular environment into the periplasmic space, attached to the outer membrane, or into the extracellular broth.

### Heterolougous Polypeptide

In some embodiments, the invention is directed to a protein comprising (i) a signal polypeptide comprising an amino acid sequence having at least 90% sequence identity to SEQ ID NO:1, and (ii) a heterologous polypeptide. In some embodiments, the invention is directed to a protein comprising (i) a signal polypeptide comprising an amino acid sequence having at least 95% sequence identity to SEQ ID NO:1, and (ii) a heterologous polypeptide. In some embodiments, the invention is directed to a protein comprising (i) a signal polypeptide comprising an amino acid sequence of SEQ ID NO:1, and (ii) a heterologous polypeptide.

As used herein, a “heterologous polypeptide,” “desired polypeptide,” “heterologous polypeptide/protein” or “polypeptide of interest” can be any polypeptide or protein, including naturally-occurring and non-naturally occurring polypeptides or proteins. As used herein, the terms “protein” and “polypeptide” are synonymous, however, for convenience, in some instances, the term “protein” is used to refer to a “heterologous polypeptide” which comprises a signal polypeptide that has not be cleaved. Heterologous polypeptides can refer to complete or partial proteins, and both functional and non-functional proteins. “Peptides” are defined as fragments or portions of polypeptides, preferably fragments or portions having at least one functional activity as the complete polypeptide sequence.

As used herein for heterologous polypeptide, “polypeptide” includes peptides having lengths of at least three amino acid residues. In some embodiments, a polypeptide has a length of at least about 10 amino acid resides, for example at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100 amino acid residues, including ranges between any two of the listed values. In some embodiments, the heterologous polypeptide comprises greater than 20 amino acid residues, greater than 30 amino acid residues, greater than 40 amino acid residues, greater than 50 amino acid residues, greater than 60 amino acid residues, greater than 70 amino acid residues, greater than 80 amino acid residues, greater than 90 amino acid residues, greater than 100 amino acid residues, greater than 120 amino acid residues, greater than 150 amino acid residues, greater than 200 amino acid residues, greater than 250 amino acid residues, greater than 300 amino acids. In some embodiments, the heterologous polypeptide comprises greater than 20 amino acids.

In some embodiments, the heterologous polypeptide comprises less than 20 amino acid residues, less than 30 amino acid residues, less than 40 amino acid residues, less than 50 amino acid residues, less than 60 amino acid residues, less than 70 amino acid residues, less than 80 amino acid residues, less than 90 amino acid residues, less than 100 amino acid residues, less than 120 amino acid residues, less than 150 amino acid residues, less than 200 amino acid residues, less than 250 amino acid residues, less than 300 amino acid residues, less than 400 amino acid residues, or less than 500 amino acids. In some embodiments, the heterologous polypeptide comprises less than 50 amino acids.

In some embodiments, the heterologous polypeptide comprises between 10 and 300 amino acid residues, between 10 and 200 amino acid residues, between 10 and 100 amino acid residues, between 10 and 50 amino acid residues, between 15 and 40 amino acid residues, between 20 and 40 amino acids, between 25 and 50 amino acids. In some embodiments, the heterologous polypeptide comprises between 25 and 40 amino acids.

In some embodiments, the heterologous polypeptide has a molecular weight of 5 kDa to 200 kDa, 10 kDa to 150 kDa, 15 kDa to 120 kDa, 20 kDa to 100 kDa, 25 kDa to 75 kDa, 25 kDa to 50 kDa, 30 kDa to 40 kDa or 30 kDa to 35 kDa. In some embodiments, the heterologous polypeptide has a molecular weight of 25 kDa to 50 kDa, or 30 kDa to 35 kDa. The term “polypeptide” further includes proteins.

In some embodiments, the heterologous polypeptides can be isolated. “Isolated” proteins or polypeptides are proteins or polypeptides purified to a state beyond that in which they exist in cells. In certain embodiments, they may be at least 10% pure; in others, they may be substantially purified to 80% or 90% purity or greater. Isolated proteins or polypeptides include essentially pure proteins or polypeptides, proteins or polypeptides produced by chemical synthesis or by combinations of biological and chemical methods, and recombinant proteins or polypeptides that are isolated. Proteins or polypeptides referred to herein as “recombinant” are proteins or polypeptides produced by the expression of recombinant nucleic acids.

Heterologous polypeptides of the present invention may be naturally occurring polypeptides. Alternatively, the heterologous polypeptides may include up to a certain integer number of amino acid alterations. Such protein or polypeptide variants retain functionality, and include mutants differing by the addition, deletion or substitution of one or more amino acid residues, or modified polypeptides and mutants comprising one or more modified residues. The variant may have one or more conservative changes, wherein a substituted amino acid has similar structural or chemical properties (e.g., replacement of leucine with isoleucine). Alterations may occur at the amino- or carboxy-terminal positions of the reference polypeptide sequence or anywhere between those terminal positions, interspersed either individually among the amino acids in the reference sequence or in one or more contiguous groups within the reference sequence.

In certain embodiments, the variant polypeptides maybe at least about 80%, 81%, 82%, 83%, 84%, 85%, 86%, 87%, 88%, 89%, 90%, 91%, 92%, 93%, 94%, 95%, 96%, 97%, 98% or 99%, identical to their naturally occurring polypeptides. Percent sequence identity can be calculated using computer programs (such as the BLASTP and TBLASTN programs publicly available from NCBI and other sources) or direct sequence comparison. Polypeptide variants can be produced using techniques known in the art including direct modifications to isolated polypeptides, direct synthesis, or modifications to the nucleic acid sequence encoding the polypeptide using, for example, recombinant DNA techniques.

Modified heterologous polypeptides, including those with post-translational modifications, are also contemplated herein. Isolated polypeptides may be modified by, for example, phosphorylation, methylation, farnesylation, carboxymethylation, geranyl geranylation, glycosylation, acetylation, myristoylation, prenylation, palmitation, amidation, sulfation, acylation, or other protein modifications. They may also be modified with a label capable of providing a detectable signal, either directly or indirectly, including, but not limited to, radioisotopes and fluorescent compounds. The polypeptides may be useful as antigens for preparing antibodies by standard methods.

These heterologous polypeptides may be fused to a signal polypeptide according the present disclosure (using, for example, recombinant technology) to direct the secretion of the heterologous polypeptide from a host cell. In some embodiments, the heterologous polypeptides may be fused to a signal polypeptide, but have a spacer polypeptide between the signal polypeptide and the heterologous polypeptide. The fusion of the signal polypeptide to the heterologous polypeptide can be accomplished by methods known to those of skill in the art, e.g., using recombinant technology. In this context, the heterologous polypeptide is referred to as the “secreted polypeptide” once it is transported to the periplasm or extracellular environment, and may include a complete polypeptide or a functional domain of a polypeptide. Any heterologous polypeptide desired to be secreted from a host cell (e.g., an enzyme or pharmaceutically active protein, etc.) is suitable for use with the signal polypeptides described herein. Signal polypeptides are typically fused to the amino terminus of a secreted polypeptide. Fused polypeptides may be produced by culturing a recombinant cell transfected with a fusion nucleic acid molecule that encodes a signal polypeptide attached to the amino terminal end of the secreted polypeptide or domain thereof. In certain embodiments, the fused signal polypeptide may also increase the expression of the secreted heterologous polypeptide in addition to directing its secretion.

As used herein, “secreted” and the like refer to a polypeptide produced by a cell, transported across or through a membrane, and exported by that cell to the periplasm, outer membrane or the extracellular environment of the cell in which it is expressed. In some embodiments, “secreted” proteins include without limitation proteins which are wholly secreted (e.g., soluble proteins) from the cell in which they are expressed. In some embodiments, the polypeptide is not stably attached to the cell. In some embodiments, a secreted heterologous polypeptide is soluble in the extracellular environment. In some embodiments, a secreted heterologous polypeptide does not include an anchor.

The extracellular presence of secreted proteins may be detected by any assay known in the art to detect a protein of interest. Examples include enzymatic activity assays, detection with specific antibodies (immunoblotting, ELISA, etc.), and other suitable detection techniques.

In some embodiments, the present invention comprises a protein comprises (i) a polypeptide comprising an amino acid having at least 90%, 95% or 100% sequence identity to SEQ ID NO:1, and (ii) a heterologous polypeptide. In some embodiments, proteins encompassed by the present invention are biologically active, that is they continue to possess the desired biological activity of the heterologous polypeptide with or without the signal polypeptide attached. By “retains activity” is intended that the protein will have at least about 30%, at least about 50%, at least about 70%, at least about 80%, about 90%, about 95%, about 100%, about 110%, about 125%, about 150%, at least about 200% or greater activity of the heterologous polypeptide.

In some embodiments, the heterologous polypeptide/protein is produced in an active form. The term “active” means the presence of biological activity, wherein the biological activity is comparable or substantially corresponds to the biological activity of a corresponding native protein or polypeptide. In the context of proteins this typically means that a polypeptide comprises a biological function or effect that has at least about 20%, about 50%, preferably at least about 60-80%, and most preferably at least about 90-95% activity compared to the corresponding native protein or polypeptide using standard parameters. The determination of protein or polypeptide activity can be performed utilizing corresponding standard, targeted comparative biological assays for particular proteins or polypeptides. One indication that a protein or polypeptide of interest maintains biological activity is that the polypeptide is immunologically cross reactive with the native polypeptide.

The methods and compositions of the present invention are useful for producing high levels of properly processed heterologous polypeptide/protein of interest in a cell expression system. The protein or polypeptide of interest can be of any species and of any size. However, in certain embodiments, the heterologous polypeptide/protein is a therapeutically useful protein or polypeptide. In some embodiments, the heterologous polypeptide/protein can be a mammalian protein, for example a human protein, and can be, for example, a growth factor, a cytokine, a chemokine or a blood protein. The heterologous polypeptide/protein can be processed in a similar manner to the native protein or polypeptide. In certain embodiments, the heterologous polypeptide is fused to signal polypeptide. In some embodiments, the protein as described herein includes a signal polypeptide.

The heterologous polypeptide of the present invention can include any polypeptide which is advantageously translocated to the periplasm or extracellular environment. In some embodiments, the heterologous polypeptide is a commercially important polypeptide. In some embodiments, the heterologous polypeptide is selected from the group consisting of an enzyme, toxin, antibody, antibody fragment, antigen, therapeutic protein, and combinations thereof. Additional examples of desired heterologous polypeptides/proteins include, but are not limited to, luciferases; fluorescent proteins (e.g., GFP); growth hormones (GHs) and variants thereof; insulin-like growth factors (IGFs) and variants thereof granulocyte colony-stimulating factors (G-CSFs) and variants thereof; erythropoietin (EPO) and variants thereof insulin, such as proinsulin, preproinsulin, insulin, insulin analogs, and the like; antibodies and variants thereof, such as hybrid antibodies, chimeric antibodies, humanized antibodies, monoclonal antibodies; antigen binding fragments of an antibody (Fab fragments), single-chain variable fragments of an antibody (scFV fragments); dystrophin and variants thereof; clotting factors and variants thereof; cystic fibrosis transmembrane conductance regulator (CFTR) and variants thereof; and interferons and variants thereof, and the like. In some embodiments, the heterologous polypeptide is an antigen used in a vaccine.

Examples of heterologous polypeptides/proteins according to the invention can include molecules such as, e.g., renin, a growth hormone, including human growth hormone; bovine growth hormone; growth hormone releasing factor; parathyroid hormone; thyroid stimulating hormone; lipoproteins; α-1-antitrypsin; insulin A-chain; insulin B-chain; proinsulin; thrombopoietin; follicle stimulating hormone; calcitonin; luteinizing hormone; glucagon; clotting factors such as factor VIIIC, factor IX, tissue factor, and von Willebrands factor; anti-clotting factors such as Protein C; atrial naturietic factor; lung surfactant; a plasminogen activator, such as urokinase or human urine or tissue-type plasminogen activator (t-PA); bombesin; thrombin; hemopoietic growth factor; tumor necrosis factor-α and 13; enkephalinase; a serum albumin such as human serum albumin; mullerian-inhibiting substance; relaxin A-chain; relaxin B-chain; prorelaxin; mouse gonadotropin-associated polypeptide; a microbial protein, such as beta-lactamase; Dnase; inhibin; activin; vascular endothelial growth factor (VEGF); receptors for hormones or growth factors; integrin; protein A or D; rheumatoid factors; a neurotrophic factor such as brain-derived neurotrophic factor (BDNF), neurotrophin-3, -4, -5, or -6 (NT-3, NT-4, NT-5, or NT-6), or a nerve growth factor such as NGF-13; cardiotrophins (cardiac hypertrophy factor) such as cardiotrophin-1 (CT-1); platelet-derived growth factor (PDGF); fibroblast growth factor such as aFGF and bFGF; epidermal growth factor (EGF); transforming growth factor (TGF) such as TGF-α and TGF-β, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-19; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-C SF, and G-CSF; interleukins (ILs), e.g., IL-1 to IL-10; anti-HER-2 antibody; superoxide dismutase; T-cell receptors; surface membrane proteins; decay accelerating factor; viral antigen such as, for example, a portion of the AIDS envelope; transport proteins; homing receptors; addressins; regulatory proteins; antibodies; and fragments of any of the above-listed polypeptides.

In certain embodiments, the heterologous polypeptide/protein can be selected from IL-1, IL-1a, IL-1b, IL-2, IL-3, IL-4, IL-5, IL-6, IL-7, IL-8, IL-9, IL-10, IL-11, IL-12, IL-12elasti, IL-13, IL-15, IL-16, IL-18, IL-18BPa, IL-23, IL-24, VIP, erythropoietin, GM-CSF, G-CSF, M-CSF, platelet derived growth factor (PDGF), MSF, FLT-3 ligand, EGF, fibroblast growth factor (FGF; e.g., α-FGF (FGF-1), β-FGF (FGF-2), FGF-3, FGF-4, FGF-5, FGF-6, or FGF-7), insulin-like growth factors (e.g., IGF-1, IGF-2); tumor necrosis factors (e.g., TNF, Lymphotoxin), nerve growth factors (e.g., NGF), vascular endothelial growth factor (VEGF); interferons (e.g., IFN-α, IFNβ, IFN-γ); leukemia inhibitory factor (LIF); ciliary neurotrophic factor (CNTF); oncostatin M; stem cell factor (SCF); transforming growth factors (e.g., TGF-α, TGF-β1, TGF-β2, TGF-γ); TNF superfamily (e.g., LIGHT/TNFSF14, STALL-1/TNFSF13B (BLy5, BAFF, THANK), TNFalpha/TNFSF2 and TWEAK/TNFSF12); or chemokines (BCA-1/BLC-1, BRAK/Kec, CXCL16, CXCR3, ENA-78/LIX, Eotaxin-1, Eotaxin-2/MPIF-2, Exodus-2/SLC, Fractalkine/Neurotactin, GROalpha/MGSA, HCC-1, I-TAC, Lymphotactin/ATAC/SCM, MCP-11MCAF, MCP-3, MCP-4, MDC/STCP-1/ABCD-1, MIP-4/PARC/DC-CK1, PF-4, RANTES, SDF1, TARC, or TECK).

In some embodiments of the present invention, the heterologous polypeptide/protein can be all or part of a multi-subunit protein or polypeptide. Multisubunit proteins that can be expressed include homomeric and heteromeric proteins. The multisubunit proteins may include two or more subunits, that may be the same or different. For example, the heterologous polypeptide/protein may be a homomeric protein comprising 2, 3, 4, 5, 6, 7, 8, 9, 10, 11, 12 or more subunits. The heterologous polypeptide/protein also may be a heteromeric protein including 2, 3, 4, 5, 6, 7, 8, 9, 10, 11, 12, or more subunits. Exemplary multisubunit proteins include: receptors including ion channel receptors; extracellular matrix proteins including chondroitin; collagen; immunomodulators including MHC proteins, full chain antibodies, and antibody fragments; enzymes including RNA polymerases, and DNA polymerases; and membrane proteins.

In another embodiment, the heterologous polypeptide/protein can be a blood protein. The blood proteins expressed in this embodiment include but are not limited to carrier proteins, such as albumin, including human and bovine albumin, transferrin, recombinant transferrin half-molecules, haptoglobin, fibrinogen and other coagulation factors, complement components, immunoglobulins, enzyme inhibitors, precursors of substances such as angiotensin and bradykinin, insulin, endothelin, and globulin, including alpha, beta, and gamma-globulin, and other types of proteins, polypeptides, and fragments thereof found primarily in the blood of mammals. The amino acid sequences for numerous blood proteins have been reported (see, S. S. Baldwin (1993) Comp. Biochem Physiol. 106b:203-218), including the amino acid sequence for human serum albumin (Lawn, L. M., et al., Nucleic Acids Research 9:6103-6114 (1981)) and human serum transferrin (Yang, F. et al., Proc. Natl. Acad. Sci. USA 81:2752-2756 (1984)).

In another embodiment, the heterologous polypeptide/protein can be a recombinant enzyme or co-factor. The enzymes and co-factors expressed in this embodiment include but are not limited to aldolases, amine oxidases, amino acid oxidases, aspartases, B12 dependent enzymes, carboxypeptidases, carboxyesterases, carboxylyases, chemotrypsin, CoA requiring enzymes, cyanohydrin synthetases, cystathione synthases, decarboxylases, dehydrogenases, alcohol dehydrogenases, dehydratases, diaphorases, dioxygenases, enoate reductases, epoxide hydrases, fumerases, galactose oxidases, glucose isomerases, glucose oxidases, glycosyltrasferases, methyltransferases, nitrile hydrases, nucleoside phosphorylases, oxidoreductases, oxynitilases, peptidases, glycosyltrasferases, peroxidases, enzymes fused to a therapeutically active polypeptide, tissue plasminogen activator; urokinase, reptilase, streptokinase; catalase, superoxide di smutase; Dnase, amino acid hydrolases (e.g., asparaginase, amidohydrolases); carboxypeptidases; proteases, trypsin, pepsin, chymotrypsin, papain, bromelain, collagenase; neuramimidase; lactase, maltase, sucrase, and arabinofuranosidases.

In another embodiment, the heterologous polypeptide/protein can be a single chain, Fab fragment and/or full chain antibody or fragments or portions thereof. A single-chain antibody can include the antigen-binding regions of antibodies on a single stably-folded polypeptide chain. Fab fragments can be a piece of a particular antibody. The Fab fragment can contain the antigen binding site. The Fab fragment can contain 2 chains: a light chain and a heavy chain fragment. These fragments can be linked via a linker or a disulfide bond.

In another embodiment, the heterologous polypeptide/protein can be an antigen used in a vaccine, e.g., a commercially available vaccine. For example, in some embodiments, the heterologous polypeptide/protein is the predominant antigen found in diphtheria, clostridium, tetanus, pertussis, polio, hepatitis B, haemphilus influenza type b, hepatitis A, rotavirus, pneumococcal, mumps, measles, rubella, varicella, human papilloma virus, meningococcal, adenovirus type 4, adenovirus type 7, anthrax, polio, meningococcal, rabies, toavirus, yellow fever, zoster, and/or influenza vaccines.

In some embodiments, the heterologous polypeptide/protein is the pore-forming α-hemolysin, also known as alpha-toxin (AT) (Natale et al., Biochimica et Biophysica Acta 1778:1735-1756 (2008). AT is produced by the majority of Staphylococcus aureus (S. aureus) and secreted as a water soluble monomer. The AT polypeptide is processed to yield a mature extracellular protein of 293 amino acids weighing approximately 33 kDa (Berube et al., Toxins 5:1140-1166 (2013)), which is one of the most well-characterized virulence factors. This protein is capable of binding and oligomerization into a heptameric structure on the host cell membrane, however it lost hemolytic activity in vitro and for lethality in an intraperitoneal murine model by substitution of histidine 35 with leucine (AT_{H35L}) (Menzies et al., Infect Immun 62:1843-1847 (1994)). Since there are no vaccines available for the prevention of S. aureus infections, a partially attenuated Alpha toxin protein (AT_{H35L}) has been studied as a vaccine target for the prevention of S. aureus infections. (Adhikari et al., PLoS ONE 7:e38567 (2012); Menzies et al, Infect Immun 64:1839-1842 (1996); Ragle et al., Infect Immun 77:2712-2718 (2009). In some embodiments, the heterologous polypeptide/protein comprises Alpha Toxin from Staphylococcus aureus, e.g., SEQ ID NO:3. In some embodiments, the Alpha Toxin comprises a substitution at amino acid position corresponding to H35. In some embodiments, the Alpha Toxin comprises an H35L substitution (AT_{H35L}), e.g., SEQ ID NO: 4. In some embodiments, the heterologous polypeptide/protein comprises AT_{H35L} and the signal polypeptide of the present invention, e.g., SEQ ID NO:2.

In certain embodiments, the heterologous polypeptide is, or is substantially homologous to, a native protein, such as a native mammalian or human protein. In these embodiments, the protein is not found in a concatameric form, but is linked only to a signal polypeptide and optionally a tag sequence for purification and/or recognition.

In some embodiments, the invention is directed to a compositions comprising the signal polypeptide as described herein and a heterologous polypeptide. In some embodiments, the composition is a medicament or therapeutic agent. In some embodiments, the composition is therapeutically effective. In some embodiments, then composition is pharmaceutically acceptable.

### Nucleic Acids

“Nucleic acid” or “polynucleotide” as used herein refers to purine- and pyrimidine-containing polymers of any length, either polyribonucleotides or polydeoxyribonucleotide or mixed polyribo-polydeoxyribonucleotides. This includes single- and double-stranded molecules (i.e., DNA-DNA, DNA-RNA and RNA-RNA hybrids). This also includes nucleic acids containing modified bases.

Nucleic acids referred to herein as “isolated” are nucleic acids that have been removed from their natural milieu or separated away from the nucleic acids of the genomic DNA or cellular RNA of their source of origin (e.g., as it exists in cells or in a mixture of nucleic acids such as a library), and may have undergone further processing. Isolated nucleic acids include nucleic acids obtained by methods described herein, similar methods or other suitable methods, including essentially pure nucleic acids, nucleic acids produced by chemical synthesis, by combinations of biological and chemical methods, and recombinant nucleic acids that are isolated. In some embodiments, any of the nucleic acids described herein may be isolated.

Nucleic acids referred to herein as “recombinant” are nucleic acids which have been produced by recombinant DNA methodology, including those nucleic acids that are generated by procedures that rely upon a method of artificial replication, such as the polymerase chain reaction (PCR) and/or cloning into a vector using restriction enzymes. Recombinant nucleic acids also include those that result from recombination events that occur through the natural mechanisms of cells, but are selected for after the introduction to the cells of nucleic acids designed to allow or make probable a desired recombination event. In some embodiments, the nucleic acids described herein can be recombinant nucleic acids.

A nucleic acid can be isolated from its natural source or produced using recombinant DNA technology (e.g., polymerase chain reaction (PCR) amplification, cloning) or chemical synthesis. Nucleic acid molecules can include, for example, genes, natural allelic variants of genes, coding regions or portions thereof, and coding and/or regulatory regions modified by nucleotide insertions, deletions, substitutions, and/or inversions in a manner such that the modifications do not substantially interfere with the nucleic acid molecule's ability to encode a polypeptide or to form stable hybrids under stringent conditions with natural gene isolates. A nucleic acid molecule can include degeneracies. As used herein, nucleotide degeneracy refers to the phenomenon that one amino acid can be encoded by different nucleotide codons. Thus, the nucleic acid sequence of a nucleic acid molecule that encodes a protein or polypeptide can vary due to degeneracies.

A nucleic acid molecule is not required to encode a polypeptide or protein having protein activity. A nucleic acid molecule can encode a truncated, mutated or inactive protein, for example.

Nucleic acids may be derived from a variety of sources including DNA, cDNA, synthetic DNA, synthetic RNA, or combinations thereof. Such sequences may comprise genomic DNA, which may or may not include naturally occurring introns. The sequences, genomic DNA, or cDNA may be obtained in any of several ways. Genomic DNA can be extracted and purified from suitable cells by means well known in the art. Alternatively, mRNA can be isolated from a cell and used to produce cDNA by reverse transcription or other means.

In some embodiments, the invention also includes a nucleic acid with a sequence that encodes a novel signal polypeptide useful for targeting an operably linked heterologous polypeptide of interest to the periplasm (of Gram-negative bacteria) or into the extracellular space. In some embodiments, the nucleic acid encodes a signal polypeptide comprising an amino acid sequence having at least 90%, 95% or 100% sequence identity to SEQ ID NO: 1. In another embodiment, the nucleic acid sequence encodes a signal polypeptide comprising an amino acid sequence having at least about 90%, 91%, 92%, 93%, 94%, 95%, 96%, 97%, 98% or 99% identical to the sequence of SEQ ID NO:1.

In some embodiments, the invention includes an isolated nucleic acid with a sequence that encodes the novel signal polypeptide described herein operably linked to AT_{H35L} (SEQ ID NO:2), which translocates the AT_{H35L} to the periplasm (of Gram-negative bacteria) or into the extracellular space. In some embodiments, the nucleic acid encodes a polypeptide comprising an amino acid sequence having at least 90%, 95% or 100% sequence identity to SEQ ID NO:2. In another embodiment, the nucleic acid encodes a polypeptide comprising an amino acid sequence having at least about 90%, 91%, 92%, 93%, 94%, 95%, 96%, 97%, 98% or 99% sequence identity to the sequence of SEQ ID NO:2.

In some embodiments, the nucleic acid comprises the signal polypeptide described herein, and one or more features to aid in cloning the nucleic acid into a vector. In some embodiments, the nucleic acid comprises one or more restriction enzyme sites.

The skilled artisan will appreciate that changes can be introduced by mutation into the nucleotide sequences of the invention that do not lead to a change in the encoded signal polypeptide. In some embodiments, the skilled artisan can introduce by mutation into the nucleotide sequences of the invention that leads to changes in the amino acid sequence of the encoded signal polypeptides, without altering the biological activity of the signal polypeptides. Thus, variant isolated nucleic acid molecules can be created by introducing one or more nucleotide substitutions, additions, or deletions into the corresponding nucleotide sequence disclosed herein, such that one or more amino acid substitutions, additions or deletions are introduced into the encoded protein. Mutations can be introduced by standard techniques, such as site-directed mutagenesis and PCR-mediated mutagenesis. Such variant nucleotide sequences are also encompassed by the present invention.

“Variant” polynucleotides of the invention include naturally-occurring polynucleotides as well as chemically altered polynucleotides. Naturally-occurring polynucleotide variants of the invention are those that (i) are found in nature, e.g., in related viral and non-viral species, (ii) are related to a polynucleotide of the invention through chemical similarity as described herein and (iii) encode a polypeptide as described herein to which the encoded polypeptide is linked. In some embodiments, the nucleic acid can comprise one or more variant polynucleotides.

The nucleic acids disclosed herein may be adjusted based on the codon usage of a host organism. Codon usage or codon preference is well known in the art. The selected coding sequence may be modified by altering the genetic code thereof to match that employed by the bacterial host cell, and the codon sequence thereof may be enhanced to better approximate that employed by the host. Genetic code selection and codon frequency enhancement may be performed according to any of the various methods known to one of ordinary skill in the art, e.g., oligonucleotide-directed mutagenesis. Useful on-line InterNet resources to assist in this process include, e.g.: (1) the Codon Usage Database of the Kazusa DNA Research Institute (2-6-7 Kazusa-kamatari, Kisarazu, Chiba 292-0818 Japan) and available at www.kazusa.orjp/codon; and (2) the Genetic Codes tables available from the NCBI Taxonomy database at www.ncbi.nln.nih.gov/-Taxonomy/Utils/wprintgc.cgi?mode=c. It is recognized that the coding sequence for either the signal polypeptide, a heterologous polypeptide of interest described elsewhere herein, or both, can be adjusted for codon usage.

### Expression Vectors

Also disclosed herein are vectors, including expression vectors, containing the signal polypeptides and heterologous polypeptides of the present invention. A “vector” or “recombinant vector” is a nucleic acid molecule that is used as a tool for manipulating a nucleic acid sequence of choice or for introducing such a nucleic acid sequence into a host cell. A recombinant vector may be suitable for use in cloning, sequencing, or otherwise manipulating the nucleic acid sequence of choice, such as by expressing or delivering the nucleic acid sequence of choice into a host cell to form a recombinant cell. Such a vector typically contains heterologous nucleic acid sequences not naturally found adjacent to a nucleic acid sequence of choice, although the vector can also contain regulatory nucleic acid sequences (e.g., promoters, untranslated regions) that are naturally found adjacent to the nucleic acid sequences of choice or that are useful for expression of the nucleic acid molecules.

A “vector” or “expression vector” is a replicon, such as a plasmid, phage, virus, transposon, phagemid, or cosmid, to which another DNA segment, i.e. an “insert”, may be attached so as to bring about the replication of the attached DNA segment in a cell. “Vector” includes episomal (e.g., plasmids) and non episomal vectors. In some embodiments of the present disclosure, the vector is an episomal vector. In some embodiments, the vector is a plasmid. In general, the vector can contain an origin of replication functional in at least one organism, convenient restriction endonuclease sites, and a selectable marker for the host cell.

A vector can be either RNA or DNA. The vector can be maintained as an extrachromosomal element (e.g., a plasmid) or it can be integrated into the chromosome of a recombinant host cell. The entire vector can remain in place within a host cell, or under certain conditions, the plasmid DNA can be deleted, leaving behind the nucleic acid molecule of choice. An integrated nucleic acid molecule can be under chromosomal promoter control, under native or plasmid promoter control, or under a combination of several promoter controls. Single or multiple copies of the nucleic acid molecule can be integrated into the chromosome. A recombinant vector can contain at least one selectable marker.

The term “expression vector” refers to a vector that is capable of directing the expression of a nucleic acid sequence that has been cloned into it after insertion into a host cell or other (e.g., cell-free) expression system. A nucleic acid sequence is “expressed” when it is transcribed to yield an mRNA sequence. In most cases in the present invention, this transcript can be translated to yield an amino acid sequence, e.g., a heterologous polypeptide/protein of the present invention. The cloned gene is usually placed under the control of (i.e., operably linked to) an expression control sequence. The phrase “operatively linked” refers to linking a nucleic acid molecule to an expression control sequence in a manner such that the molecule can be expressed when introduced (i.e., transformed, transduced, transfected, conjugated or conduced) into a host cell.

Recombinant vectors and expression vectors may contain one or more regulatory sequences or expression control sequences. Regulatory sequences broadly encompass expression control sequences (e.g., transcription control sequences or translation control sequences), as well as sequences that allow for vector replication in a host cell. Transcription control sequences are sequences that control the initiation, elongation, or termination of transcription. Suitable regulatory sequences include any sequence that can function in a host cell or organism into which the recombinant nucleic acid molecule is to be introduced, including those that control transcription initiation, such as promoter, enhancer, terminator, operator and repressor sequences. Additional regulatory sequences include translation regulatory sequences, origins of replication, and other regulatory sequences that are compatible with the recombinant cell (see, e.g., D. V. Goeddel, Methods Enzymol. 185:3-7).

The expression vectors may contain elements that allow for constitutive expression or inducible expression of the protein or proteins of interest. As used herein, “promoter,” “promoter sequence,” or promoter region” refers to a DNA regulatory region/sequence capable of binding RNA polymerase and involved in initiating transcription of a downstream coding or non-coding sequence. In some examples of the present disclosure, the promoter sequence includes the transcription initiation site and extends upstream to include the minimum number of bases or elements necessary to initiate transcription at levels detectable above background. In some embodiments, the promoter sequence includes a transcription initiation site, as well as protein binding domains responsible for the binding of RNA polymerase. Various promoters, including inducible promoters, may be used to drive the various vectors of the present invention.

Numerous inducible and constitutive expression systems are known in the art. For example, expression control sequences such as promoter regions can be selected from any desired gene. Bacterial promoters may include lad, lacZ, T3, T7, gpt, lambda PR, Ptac16, Ptac17, PtacII, PlacUV5, T7lac promoter and trc. In some embodiments, vectors comprising the Ptac promoter allow for constitutive expression in the absence of the lad gene, but expression may be induced by the addition of isopropyl-β-D-thiogalactopyranoside (IPTG) when the vector also contains the lad gene. Selection of the appropriate promoter is well within the level of ordinary skill in the art.

The promoters used in accordance with the present invention may be constitutive promoters or regulated promoters. In some embodiments, the promoter is not derived from the host cell organism. In certain embodiments, the promoter is derived from an E. coli organism. Common examples of non-lac-type promoters useful in expression systems according to the present invention are known to the skilled artisan. See, e.g.: J. Sanchez-Romero & V. De Lorenzo (1999) Genetic Engineering of Nonpathogenic Pseudomonas strains as Biocatalysts for Industrial and Environmental Processes, in Manual of Industrial Microbiology and Biotechnology (A. Demain & J. Davies, eds.) pp. 460-74 (ASM Press, Washington, D.C.); H. Schweizer (2001) Vectors to express foreign genes and techniques to monitor gene expression for Pseudomonads, Current Opinion in Biotechnology, 12:439-445; and R. Slater & R. Williams (2000) The Expression of Foreign DNA in Bacteria, in Molecular Biology and Biotechnology (J. Walker & R. Rapley, eds.) pp. 125-54 (The Royal Society of Chemistry, Cambridge, UK)). A promoter having the nucleotide sequence of a promoter native to the selected bacterial host cell may also be used to control expression of the transgene encoding the target polypeptide, e.g., a Pseudomonas anthranilate or benzoate operon promoter (Pant, Pben). Tandem promoters may also be used in which more than one promoter is covalently attached to another, whether the same or different in sequence, e.g., a Pant-Pben tandem promoter (interpromoter hybrid) or a Plac-Plac tandem promoter, or whether derived from the same or different organisms.

Regulated promoters utilize promoter regulatory proteins in order to control transcription of the gene of which the promoter is a part. Where a regulated promoter is used herein, a corresponding promoter regulatory protein will also be part of an expression system according to the present invention. Examples of promoter regulatory proteins include: activator proteins, e.g., E. coli catabolite activator protein, MalT protein; AraC family transcriptional activators; repressor proteins, e.g., E. coli Lad proteins; and dual-function regulatory proteins, e.g., E. coli NagC protein. Many regulated-promoter/promoter-regulatory-protein pairs are known in the art.

Promoter regulatory proteins interact with an effector compound, i.e. a compound that reversibly or irreversibly associates with the regulatory protein so as to enable the protein to either release or bind to at least one DNA transcription regulatory region of the gene that is under the control of the promoter, thereby permitting or blocking the action of a transcriptase enzyme in initiating transcription of the gene. Effector compounds are classified as either inducers or co-repressors, and these compounds include native effector compounds and gratuitous inducer compounds. Many regulated-promoter/promoter-regulatory-protein/effector-compound trios are known in the art. Although an effector compound can be used throughout the cell culture or fermentation, in a preferred embodiment in which a regulated promoter is used, after growth of a desired quantity or density of host cell biomass, an appropriate effector compound is added to the culture to directly or indirectly result in expression of the desired gene(s) encoding the protein or polypeptide of interest.

By way of example, where a lac family promoter is utilized, a lad gene can also be present in the system. The lad gene, which is (normally) a constitutively expressed gene, encodes the Lac repressor protein (LacD protein) which binds to the lac operator of these promoters. Thus, where a lac family promoter is utilized, the lad gene can also be included and expressed in the expression system. In the case of the lac promoter family members, e.g., the tac promoter, the effector compound is an inducer, preferably a gratuitous inducer such as IPTG (isopropyl-D-1-thiogalactopyranoside, also called “isopropylthiogalactoside”).

In some embodiments, a pET expression system is used. pET expression system provides a high level of protein production. Expression is induced from the strong T7lac promoter. This system takes advantage of the high activity and specificity of the bacteriophage T7 RNA polymerase for high level transcription of the gene of interest. The lac operator located in the promoter region provides tighter regulation than traditional T7-based vectors, improving plasmid stability and cell viability (Studier and Moffatt, J Molecular Biology 189(1):113-30 (1986); Rosenberg, et al., Gene 56(1): 125-35 (1987)). The T7 expression system uses the T7 promoter and T7 RNA polymerase (T7 RNAP) for high-level transcription of the gene of interest. High-level expression is achieved in T7 expression systems because the T7 RNAP is more processive than native E. coli RNAP and is dedicated to the transcription of the gene of interest. Expression of the identified gene is induced by providing a source of T7 RNAP in the host cell. This is accomplished by using a BL21 E. coli host containing a chromosomal copy of the T7 RNAP gene. The T7 RNAP gene is under the control of the lacUV5 promoter which can be induced by IPTG. T7 RNAP is expressed upon induction and transcribes the gene of interest.

In some embodiments, the pBAD expression system is used. The pBAD expression system allows tightly controlled, titratable expression of protein or polypeptide of interest through the presence of specific carbon sources such as glucose, glycerol and arabinose (Guzman, et al., J Bacteriology 177(14): 4121-30 (1995)). The pBAD vectors are uniquely designed to give precise control over expression levels. Heterologous gene expression from the pBAD vectors is initiated at the araBAD promoter. The promoter is both positively and negatively regulated by the product of the araC gene. AraC is a transcriptional regulator that forms a complex with L-arabinose. In the absence of L-arabinose, the AraC dimer blocks transcription. For maximum transcriptional activation two events are required: (i.) L-arabinose binds to AraC allowing transcription to begin. (ii.) The cAMP activator protein (CAP)-cAMP complex binds to the DNA and stimulates binding of AraC to the correct location of the promoter region.

In some embodiments, the trc expression system is used. The trc expression system allows high-level, regulated expression in E. coli from the trc promoter. The trc expression vectors have been optimized for expression of eukaryotic genes in E. coli. The trc promoter is a strong hybrid promoter derived from the tryptophan (trp) and lactose (lac) promoters. It is regulated by the lacO operator and the product of the lacIQ gene (Brosius, J., Gene 27:161-72 (1984)).

In some embodiments, a pJ411 expression system is used (DNA2.0 Inc., Menlo Park, Calif., USA). Expression includes a T7 promoter and a kanamycin resistance marker.

In another embodiment, the expression vector further comprises a tag sequence adjacent to the coding sequence for the signal polypeptide or to the coding sequence for the heterologous polypeptide/protein of interest. In some embodiments, this tag sequence allows for identification, separation, purification, and/or isolation of the protein. The tag sequence can be an affinity tag, such as a hexa-histidine affinity tag. In another embodiment, the affinity tag can be a glutathione-S-transferase molecule. The tag can also be a fluorescent molecule, such as YFP or GFP, or analogs of such fluorescent proteins. The tag can also be a portion of an antibody molecule, or a known antigen or ligand for a known binding partner useful for purification. Thus, in some embodiments, the vector can comprise a nucleic acid sequence which encodes a protein comprising a signal polypeptide, a heterologous polypeptide and a tag sequence.

Other regulatory elements may be included in a vector (also termed “expression construct”). Such elements include, but are not limited to, for example, transcriptional enhancer sequences, translational enhancer sequences, other promoters, activators, translational start and stop signals, transcription terminators, cistronic regulators, and polycistronic regulators.

Typically, a vector includes at least one nucleic acid molecule encoding a signal polypeptide and a heterologous polypeptide operatively linked to one or more expression control sequences (e.g., transcription control sequences or translation control sequences). In one aspect, an expression vector may comprise a nucleic acid encoding a signal polypeptide, as described herein, fused to a nucleic acid encoding a heterologous polypeptide/protein to be expressed, and operably linked to at least one regulatory sequence. Exemplary embodiments include expression vectors comprising the nucleic acids encoding signal polypeptide as described herein fused to AT_{H35L} (SEQ ID NO:2). It should be understood that the design of the expression vector may depend on such factors as the choice of the host cell to be transformed and/or the type of polypeptide to be expressed.

Generally, vectors will include origins of replication and selectable markers which permit identification and isolation of transformed host cells, e.g., the ampicillin resistance gene of E. coli, and a promoter derived to direct transcription of a downstream structural sequence. The heterologous structural sequence is assembled in appropriate phase with translation initiation and termination sequences, and may include a signal polypeptide as described herein capable of directing secretion of translated protein into the periplasmic space or extracellular medium. Useful expression vectors for bacterial use are constructed by inserting a structural DNA sequence encoding the signal polypeptide and heterologous polypeptide together with suitable translation initiation and termination signals in operable reading phase with a functional promoter. The vector can comprise one or more phenotypic selectable markers and an origin of replication to ensure maintenance of the vector and to, if desirable, provide amplification within the host.

A vector according to the present invention can include, in addition to the protein coding sequence, a ribosome binding site (RBS), a transcription terminator, translational start and stop signals. Useful RBSs can be obtained from any of the species useful as host cells in expression systems according to the present invention, preferably from the selected host cell. Many specific and a variety of consensus RBSs are known, e.g., those described in and referenced by D. Frishman et al., Starts of bacterial genes: estimating the reliability of computer predictions, Gene 234(2):257-65 (8 Jul. 1999); and B. E. Suzek et al., A probabilistic method for identifying start codons in bacterial genomes, Bioinformatics 17(12):1123-30 (December 2001). In addition, either native or synthetic RBSs may be used, e.g., those described in: EP 0207459 (synthetic RBSs); O. Ikehata et al., Eur. J. Biochem. 181(3):563-70 (1989) (native RBS sequence of AAGGAAG). Further examples of methods, vectors, and translation and transcription elements, and other elements useful in the present invention are described in, e.g.: U.S. Pat. No. 5,055,294 to Gilroy and U.S. Pat. No. 5,128,130 to Gilroy et al.; U.S. Pat. No. 5,281,532 to Rammler et al.; U.S. Pat. Nos. 4,695,455 and 4,861,595 to Barnes et al.; U.S. Pat. No. 4,755,465 to Gray et al.; and U.S. Pat. No. 5,169,760 to Wilcox.

Transcription of the DNA encoding the proteins and heterologous polypeptides as described herein is increased by inserting an enhancer sequence into the vector or plasmid. Typical enhancers are cis-acting elements of DNA, usually about from 10 to 300 bp in size that act on the promoter to increase its transcription. Examples include various E. coli enhancers.

Generally, the vectors will include origins of replication and selectable markers permitting transformation of the host cell and a promoter derived from a highly-expressed gene to direct transcription of a downstream structural sequence. Such promoters can be derived from operons encoding the enzymes such as 3-phosphoglycerate kinase (PGK), acid phosphatase, or heat shock proteins, among others. The heterologous structural sequence is assembled in appropriate phase with translation initiation and termination sequences, and preferably, the signal polypeptide capable of directing secretion of the translated polypeptide. Optionally the heterologous sequence can encode a fusion polypeptide including an N-terminal identification polypeptide imparting desired characteristics, e.g., stabilization or simplified purification of expressed recombinant product.

Vectors may contain a selectable marker, a gene encoding a protein necessary for survival or growth of a host cell transformed with the vector. The presence of this gene allows growth of only those host cells that express the vector when grown in the appropriate selective media. Typical selection genes encode proteins that confer resistance to antibiotics or other toxic substances, complement auxotrophic deficiencies, or supply critical nutrients not available from a particular media. Markers may be an inducible or non-inducible gene and will generally allow for positive selection. Non-limiting examples of selectable markers include the ampicillin resistance marker (i.e., beta-lactamase), tetracycline resistance marker, neomycin/kanamycin resistance marker (i.e., neomycin phosphotransferase), dihydrofolate reductase, glutamine synthetase, and the like. In some embodiments, the selectable marker gene is, e.g., a prototrophy-restoring gene where the vector is used in a host cell that is auxotrophic for the corresponding trait, e.g., a biocatalytic trait such as an amino acid biosynthesis or a nucleotide biosynthesis trait, or a carbon source utilization trait. The choice of the proper selectable marker will depend on the host cell, and appropriate markers for different hosts as understood by those of skill in the art.

Large numbers of suitable vectors (many of which include endogenous regulatory DNA elements) are known to those of skill in the art and are commercially available or may be derived from commercially available vectors. As a representative but non-limiting example, useful vectors for bacterial use can comprise a selectable marker and bacterial origin of replication derived from commercially available plasmids comprising genetic elements of the well-known cloning vector pBR322 (ATCC 37017). Such commercial vectors include, for example, pKK223-3 (Pharmacia Fine Chemicals, Uppsala, Sweden) and GEM 1 (Promega Biotech, Madison, Wis., USA). These pBR322 “backbone” sections are combined with an appropriate promoter and the structural sequence to be expressed. Other exemplary bacterial vectors include, for example, pBs, phagescript, PsiX174, pBluescript SK, pBs KS, pNH8a, pNH16a, pNH18a, pNH46a (Stratagene); pTrc99A, pKK223-3, pKK233-3, pDR540, and pRIT5 (Pharmacia). Following transformation of a suitable host strain and growth of the host strain to an appropriate cell density, the selected promoter is induced or derepressed by appropriate means (e.g., temperature shift or chemical induction) and cells are cultured for an additional period.

Other examples of useful plasmid vectors include, but are not limited to, vectors as described by, e.g.: N. Hayase, in Appl. Envir. Microbiol. 60(9):3336-42 (September 1994); A. A. Lushnikov et al., in Basic Life Sci. 30:657-62 (1985); S. Graupner & W. Wackemagel, in Biomolec. Eng. 17(1):11-16. (October 2000); H. P. Schweizer, in Curr. Opin. Biotech. 12(5):439-45 (October 2001); M. Bagdasarian & K. N. Timmis, in Curr. Topics Microbiol. Immunol. 96:47-67 (1982); T. Ishii et al., in FEMS Microbiol. Lett. 116(3):307-13 (Mar. 1, 1994); I. N. Olekhnovich & Y. K. Fomichev, in Gene 140(1):63-65 (Mar. 11, 1994); M. Tsuda & T. Nakazawa, in Gene 136(1-2):257-62 (Dec. 22, 1993); C. Nieto et al., in Gene 87(1):145-49 (Mar. 1, 1990); J. D. Jones & N. Gutterson, in Gene 61(3):299-306 (1987); M. Bagdasarian et al., in Gene 16(1-3):237-47 (December 1981); H. P. Schweizer et al., in Genet. Eng. (NY) 23:69-81 (2001); P. Mukhopadhyay et al., in J. Bact. 172(1):477-80 (January 1990); D. O. Wood et al., in J. Bact. 145(3):1448-51 (March 1981); and R. Holtwick et al., in Microbiology 147(Pt 2):337-44 (February 2001).

### Host Cells

The present invention provides host cells genetically engineered to express the signal polypeptides and heterologous polypeptides proteins as described herein, wherein the nucleic acids encoding the signal polypeptides and heterologous polypeptides proteins comprise a promoter operably linked to the signal polypeptides and/or heterologous polypeptides/proteins which drives expression of the polynucleotides in the cell. The host cell can be a prokaryotic host cell, such as a gram positive or gram negative bacterial cell. In some embodiments, the promoter sequence is operable in a prokaryote.

In some embodiments, the invention is directed to a recombinant cell engineered to express the proteins, heterologous polypeptides, and/or signal polypeptides described herein. The term “recombinant cell” refers to a host cell comprising a nucleic acid sequence as described herein, wherein the nucleic acid encodes the signal polypeptide as described herein. In some embodiments, the nucleic acid encoding the signal polypeptide is on one or more vectors. In some embodiments, the nucleic acid encoding the signal polypeptide can be incorporated into the genome of the host cell. In some embodiments, the recombinant cell is a prokaryote cell. In some embodiments, the recombinant cell is of the genus Escherichia. In some embodiments, the recombinant prokaryote cell is Escherichia coli.

In some embodiments, the present invention is directed to a host cell transformed with any vector operably encoding the signal polypeptide as described herein. Transformation of the host cells with the vector(s) disclosed herein may be performed using any transformation methodology known in the art, and the bacterial host cells may be transformed as intact cells or as protoplasts (i.e. including cytoplasts). Exemplary transformation methodologies include poration methodologies, e.g., electroporation, protoplast fusion, bacterial conjugation, and divalent cation treatment, e.g., calcium chloride treatment or CaCl/Mg2+ treatment, or other well-known methods in the art. See, e.g., Morrison, J. Bact., 132:349-351 (1977); Clark-Curtiss & Curtiss, Methods in Enzymology, 101:347-362 (Wu et al., eds, 1983), Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

For example, such host cells may contain nucleic acids of the invention introduced into the host cell using known transformation, transfection or infection methods. The term “transformation” refers to the introduction of DNA into a suitable host cell so that the DNA is replicable, either as an extrachromosomal element, or by chromosomal integration. The term “transfection” as used herein refers to the taking up of an expression vector by a suitable host cell, whether or not any coding sequences are in fact expressed. The term “infection” refers to the introduction of nucleic acids into a suitable host cell by use of a virus or viral vector. Introduction of the recombinant construct into the host cell can be effected by calcium phosphate transfection, DEAE dextran mediated transfection, or electroporation (Davis, L. et al., Basic Methods in Molecular Biology (1986)).

Host cells can be transformed, transfected, or infected as appropriate by any suitable method including electroporation, calcium chloride-, Lithium chloride-, lithium acetate/polyethylene glycol-, calcium phosphate-, DEAE-dextran-, liposome-mediated DNA uptake, spheroplasting, injection, microinjection, microprojectile bombardment, phage infection, viral infection, or other established methods. Exemplary embodiments include a host cell or population of cells expressing one or more nucleic acid molecules or expression vectors described herein (for example, a genetically modified microorganism). The cells into which nucleic acids have been introduced as described above also include the progeny of such cells.

Host cells carrying a vector as described herein (i.e., transformants or clones) may be selected using markers depending on the mode of the vector construction. The marker may be on the same or a different DNA molecule. In prokaryotic hosts, the transformant may be selected, for example, by resistance to ampicillin, tetracycline or other antibiotics. Production of a particular product based on temperature sensitivity may also serve as an appropriate marker.

Host cells may be cultured in an appropriate medium. An appropriate, or effective, medium refers to any medium in which a host cell, including a genetically modified microorganism, when cultured, is capable of growing and/or expressing heterologous polypeptides/proteins of the present invention. Such a medium is typically an aqueous medium comprising carbon, nitrogen and phosphate sources, but can also include appropriate salts, minerals, metals and other nutrients. Microorganisms and other cells can be cultured in conventional bioreactors and by any process, including batch, fed-batch, cell recycle, and continuous fermentation. The pH of the culture medium is regulated to a pH suitable for growth and protein production of the particular organism. The growth chamber can be aerated in order to supply the oxygen necessary for growth and to avoid the excessive accumulation of carbon dioxide. Culture media and conditions for various host cells are known in the art.

In some embodiments, the host cell is a prokaryote cell. In some embodiments, the prokaryote is a gram negative bacteria, e.g., bacteria of the genus Escherichia, Pseudomonas, Neisseria, Yersinia, Salmonella, Shigella, Moaxella, Helocobacter, Stenotrophomonas, Bacillus, Staphylococcus, Streptomyces, Bdellovibrio, Leionella, cyanobacteria, spirochasetes, green sulfur vacteria, Klebsiella, or Serratia. In some embodiments, the gram negative bacteria is of the genus Escherichia, e.g., Escherichia coli.

In some embodiments, the host cell can be any cell capable of producing a protein or polypeptide of interest, including any one of the gram negative prokaryotes as described above. The most commonly used systems to produce proteins or polypeptides of interest include certain bacterial cells, particularly E. coli, because of their relatively inexpensive growth requirements and potential capacity to produce protein in large batch cultures. These systems are well characterized, provide generally acceptable levels of total protein expression and are comparatively fast and inexpensive. Typical bacterial cells are described, for example, in “Biological Diversity: Bacteria and Archaeans”, a chapter of the On-Line Biology Book, provided by Dr M J Farabee of the Estrella Mountain Community College, Arizona, USA at the website www.emc.maricotpa.edu/faculty/farabee/BIOBK/BioBookDiversity. In certain embodiments, the host cell can be an Escherichia cell, and can typically be an Escherichia coli cell.

In some embodiments, the host cell can be a member of any of the bacterial taxa. The cell can, for example, be a member of any species of eubacteria. The host can be a member of any one of the taxa: Acidobacteria, Actinobacteira, Aquificae, Bacteroidetes, Chlorobi, Chlamydiae, Choroflexi, Chrysiogenetes, Cyanobacteria, Deferribacteres, Deinococcus, Dictyoglomi, Fibrobacteres, Firmicutes, Fusobacteria, Gemmatimonadetes, Lentisphaerae, Nitrospirae, Planctomycetes, Proteobacteria, Spirochaetes, Thermodesulfobacteria, Thermomicrobia, Thermotogae, Thermus (Thermales), or Verrucomicrobia. In an embodiment of a eubacterial host cell, the cell can be a member of any species of eubacteria.

The bacterial host can also be a member of any species of Proteobacteria. A proteobacterial host cell can be a member of any one of the taxa Alphaproteobacteria, Betaproteobacteria, Gammaproteobacteria, Deltaproteobacteria, or Epsilonproteobacteria. In addition, the host can be a member of any one of the taxa Alphaproteobacteria, Betaproteobacteria, or Gammaproteobacteria, and a member of any species of Gammaproteobacteria.

In some embodiments of a Gamma Proteobacterial host, the host will be member of any one of the taxa Aeromonadales, Alteromonadales, Enterobacteriales, Pseudomonadales, or Xanthomonadales; or a member of any species of the Enterobacteriales or Pseudomonadales. In some embodiments, the host cell can be of the order Enterobacteriales, the host cell will be a member of the family Enterobacteriaceae, or may be a member of any one of the genera Envinia, Escherichia, or Serratia; or a member of the genus Escherichia. Where the host cell is of the order Pseudomonadales, the host cell may be a member of the family Pseudomonadaceae, including the genus Pseudomonas. Gamma Proteobacterial hosts include members of the species Escherichia coli and members of the species Pseudomonas fluorescens.

### Methods of Producing Secreted Heterologous Proteins

The methods of the invention provide the expression of fusion proteins comprising a signal polypeptide as described herein. In some embodiments, the method includes a host cell expressing heterologous polypeptide of interest linked to a signal polypeptide of the invention. The methods include providing a host cell, e.g., a E. coli host cell, comprising a vector encoding a recombinant protein comprising the protein or polypeptide of interest operably linked to a signal polypeptide disclosed herein, and growing the cell under conditions that result in expression of the protein or polypeptide. Alternatively, the method of expressing proteins or polypeptides using the identified signal polypeptides can be used in any given host system, including host cells of other prokaryotic origin. The vector can have any of the characteristics described above. In some embodiments, the host cell comprises a vector comprises a nucleotide sequence encoding the signal polypeptides disclosed herein as SEQ ID NO:1, or variants and fragments thereof. In another embodiment, the vector comprises a nucleotide sequence encoding the protein of SEQ ID NO:2, or variants and fragments thereof.

In some embodiments, the invention is directed to a method of producing a protein, e.g., heterologous polypeptide of the invention, comprising culturing a recombinant cell engineered to express the protein, or a host cell transformed with a vector encoding the protein, under conditions in which the protein is expressed. In some embodiments, the recombinant cell or host cell is cultured in cell culture under conditions in which the protein is secreted from the recombinant cell or host cell. In some embodiments, the recombinant cell or host cell is cultured in cell culture under conditions in which the signal polypeptide is cleaved from the protein.

In another embodiment, the host cell has a periplasm and expression of the signal polypeptide results in the targeting of substantially all of the heterologous polypeptide of interest to the periplasm of the cell. It is recognized that a small fraction of the protein expressed in the periplasm may actually leak through the cell membrane into the extracellular space; however, the majority of the targeted polypeptide would remain within the periplasmic space.

The cell growth conditions for the host cells described herein can include that which facilitates expression of the protein of interest, and/or that which facilitates fermentation of the expressed protein of interest. As used herein, the term “fermentation” includes both embodiments in which literal fermentation is employed and embodiments in which other, non-fermentative culture modes are employed. In some embodiments, the fermentation medium may be selected from among rich media, minimal media, and mineral salts media; a rich medium may be used, but is preferably avoided. In another embodiment either a minimal medium or a mineral salts medium is selected. In still another embodiment, a minimal medium is selected. In yet another embodiment, a mineral salts medium is selected. Mineral salts media are particularly preferred.

The expression system according to the present invention can be cultured in any fermentation format. For example, batch, fed-batch, semi-continuous, and continuous fermentation modes may be employed herein. Wherein the protein is excreted into the extracellular medium, continuous fermentation is preferred.

Fermentation may be performed at any scale. Thus, e.g., microliter-scale, centiliter scale, and deciliter scale fermentation volumes may be used; and 1 Liter scale and larger fermentation volumes can be used. In some embodiments, the fermentation volume will be at or above 1 Liter. In another embodiment, the fermentation volume will be at or above 5 Liters, 10 Liters, 15 Liters, 20 Liters, 25 Liters, 50 Liters, 75 Liters, 100 Liters, 200 Liters, 500 Liters, 1,000 Liters, 2,000 Liters, 5,000 Liters, 10,000 Liters or 50,000 Liters.

In the present invention, growth, culturing, and/or fermentation of the transformed host cells is performed within a temperature range permitting survival of the host cells, preferably a temperature within the range of about 4° C. to about 55° C., inclusive. Thus, e.g., the terms “growth” (and “grow,” “growing”), “culturing” (and “culture”), and “fermentation” (and “ferment,” “fermenting”), as used herein in regard to the host cells of the present invention, inherently means “growth,” “culturing,” and “fermentation,” within a temperature range of about 4° C. to about 55° C., inclusive. In addition, “growth” is used to indicate both biological states of active cell division and/or enlargement, as well as biological states in which a non-dividing and/or non-enlarging cell is being metabolically sustained, the latter use of the term “growth” being synonymous with the term “maintenance.”

The signal polypeptide can be expressed in a manner in which it is linked to the heterologous polypeptide and the signal-linked polypeptide can be purified from the cell. Therefore, in some embodiments, this isolated heterologous polypeptide is a fusion protein of the signal polypeptide and the heterologous polypeptide of interest. However, the signal polypeptide can also be cleaved from the heterologous polypeptide when the heterologous polypeptide is targeted to the periplasm. In some embodiments, the linkage between the signal polypeptide and the heterologous protein or polypeptide is modified to increase cleavage of the signal polypeptide.

The expression may lead to production of extracellular heterologous polypeptide. The method may also include the step of purifying the heterologous polypeptide of interest from the periplasm or from extracellular media. In some embodiments, the invention comprises producing the protein, e.g., heterologous polypeptide, and then recovering the protein from the cell culture. In some embodiments, recovering the protein comprises centrifugation to remove cells and/or cellular debris. In some embodiments, recovering the protein comprises filtering to remove cells and/or cellular debris.

The phrase “recovering the protein” refers to collecting the whole culture medium containing the protein and need not imply additional steps of separation or purification. Proteins can be purified using a variety of standard protein purification techniques, such as affinity chromatography, ion exchange chromatography, filtration, electrophoresis, hydrophobic interaction chromatography, gel filtration chromatography, reverse phase chromatography, concanavalin A chromatography, chromato focusing, differential solubilization, preparative disc-gel electrophoresis, isoelectric focusing, HPLC, reversed-phase HPLC, or countercurrent distribution. The polypeptide may contain an additional protein or epitope tag that facilitates detection or purification, such as c-myc, haemagglutinin (HA), polyhistidine, GLU-GLU, FLAG-tag, glutathione-S-transferase (GST), green fluorescent protein (GFP), or maltose binding protein (MBP). Such tags may be removed following the recovery of the polypeptide.

The methods of the invention may also lead to increased production of the protein or polypeptide of interest within the host cell. The increased production alternatively can be an increased level of properly processed protein or polypeptide per gram of protein produced, or per gram of host protein. The increased production can also be an increased level of recoverable protein or polypeptide produced per gram of recombinant or per gram of host cell protein. The increased production can also be any combination of an increased level of total protein, increased level of properly processed protein, or increased level of active or soluble protein. In this embodiment, the term “increased” is relative to the level of protein or polypeptide that is produced, properly processed, soluble, and/or recoverable when the protein or polypeptide of interest is expressed in a cell without the signal polypeptide of the invention, or when the level of protein or polypeptide that is produced, properly processed, soluble, and/or recoverable when the protein or polypeptide of interest is expressed with a dsbA signal polypeptide.

An improved expression of heterologous polypeptide of interest can also refer to an increase in the solubility of the polypeptide. The heterologous polypeptide of interest can be produced and recovered from the cytoplasm, periplasm or extracellular medium of the host cell. The heterologous polypeptide can be insoluble or soluble. The heterologous polypeptide can include one or more sequences to assist purification, as discussed supra.

The term “soluble” as used herein means that the heterologous polypeptide is not precipitated by centrifugation at between approximately 5,000× and 20,000× gravity when spun for 10-30 minutes in a buffer under physiological conditions. Soluble proteins are not part of an inclusion body or other precipitated mass. Similarly, “insoluble” means that the protein or polypeptide can be precipitated by centrifugation at between 5,000× and 20,000× gravity when spun for 10-30 minutes in a buffer under physiological conditions. Insoluble proteins or polypeptides can be part of an inclusion body or other precipitated mass. The term “inclusion body” is meant to include any intracellular body contained within a cell wherein an aggregate of proteins or polypeptides has been sequestered.

The methods of the invention can produce heterologous polypeptide localized to the periplasm of the host cell. In some embodiments, the method produces properly processed heterologous polypeptides of interest in the cell. In another embodiment, the expression of the signal polypeptide may produce active heterologous polypeptides of interest in the cell. The method of the invention may also lead to an increased yield of heterologous polypeptides of interest as compared to when the heterologous polypeptide is expressed without the signal polypeptide of the invention, or as compared to when the heterologous polypeptide is expressed with the dsbA signal polypeptide.

In some embodiments, the method produces at least 0.1 g/L protein in the periplasmic compartment. In another embodiment, the method produces 0.1 to 10 g/L periplasmic protein in the cell, or at least about 0.2, about 0.3, about 0.4, about 0.5, about 0.6, about 0.7, about 0.8, about 0.9 or at least about 1.0 g/L periplasmic protein. In some embodiments, the total protein or polypeptide of interest produced is at least 1.0 g/L, at least about 2 g/L, at least about 3 g/L, about 4 g/L, about 5 g/L, about 6 g/L, about 7 g/L, about 8 g/L, about 10 g/L, about 15 g/L, about 20 g/L, at least about 25 g/L, or greater. In some embodiments, the amount of periplasmic protein produced is at least about 5%, about 10%, about 15%, about 20%, about 25%, about 30%, about 40%, about 50%, about 60%, about 70%, about 80%, about 90%, about 95%, about 96%, about 97%, about 98%, about 99%, or more of total heterologous polypeptide of interest produced.

In some embodiments, the method produces at least 0.1 g/L correctly processed heterologous polypeptide. A correctly processed heterologous polypeptide has an amino terminus of the native protein. In some embodiments, at least 50% of the heterologous polypeptide of interest comprises a native amino terminus. In another embodiment, at least 60%, at least 70%, at least 80%, at least 90%, or more of the heterologous polypeptide has an amino terminus of the native protein. In various embodiments, the method produces 0.1 to 10 g/L correctly processed heterologous polypeptide in the cell, including at least about 0.2, about 0.3, about 0.4, about 0.5, about 0.6, about 0.7, about 0.8, about 0.9 or at least about 1.0 g/L correctly processed protein. In another embodiment, the total correctly processed heterologous polypeptide of interest produced is at least 1.0 g/L, at least about 2 g/L, at least about 3 g/L, about 4 g/L, about 5 g/L, about 6 g/L, about 7 g/L, about 8 g/L, about 10 g/L, about 15 g/L, about 20 g/L, about 25 g/L, about 30 g/L, about 35 g/l, about 40 g/l, about 45 g/l, at least about 50 g/L, or greater. In some embodiments, the amount of correctly processed heterologous polypeptide produced is at least about 5%, about 10%, about 15%, about 20%, about 25%, about 30%, about 40%, about 50%, about 60%, about 70%, about 80%, about 90%, about 95%, about 96%, about 97%, about 98%, at least about 99%, or more of total recombinant protein in a correctly processed form.

The methods of the invention can also lead to increased yield of heterologous polypeptide of interest. In some embodiments, the method produces a heterologous polypeptide of interest as at least about 5%, at least about 10%, about 15%, about 20%, about 25%, about 30%, about 40%, about 45%, about 50%, about 55%, about 60%, about 65%, about 70%, about 75%, or greater of total cell protein (tcp). “Percent total cell protein” is the amount of heterologous polypeptide in the host cell as a percentage of aggregate cellular protein. The determination of the percent total cell protein is well known in the art.

In a particular embodiment, the host cell can have a heterologous polypeptide, or fragment thereof, expression level of at least 1% tcp and a cell density of at least 40 g/L, when grown (i.e. within a temperature range of about 4° C. to about 55° C., including about 10° C., about 15° C., about 20° C., about 25° C., about 30° C., about 35° C., about 40° C., about 45° C., and about 50° C.).

In some embodiments, the invention is directed to increasing the rate of heterologous polypeptide secretion from a host cell. The rate of secretion in some instances can advantageously reduce production times, thereby reducing costs and increasing efficiencies. In some embodiments, the invention is directed to a method of increasing the rate of protein secretion from a cell, comprising: (a) culturing in cell culture a host cell comprising the nucleic acid or vector as described herein which encodes a protein comprising the signal polypeptide, (b) inducing expression of the protein, and (c) recovering the protein comprising the signal polypeptide secreted into the supernatant of the cell culture, wherein the rate of protein secretion is compared to the rate of protein secretion of the protein with a dsbA signal polypeptide. The rate of protein secretion can be measured by measuring the amount of protein into the extracellular environment (i.e., supernatant of the cell culture) secreted over time.

In some embodiments, the rate of protein secretion reduces the amount of time required to produce the desired quantity of protein. For example, in some embodiments, the recovering of the protein comprising the signal polypeptide secreted into the supernatant of the cell culture occurs between 6 and 15 hours, 7 and 12 hours, or 8 and 10 hours after the inducing of expression of the protein. In some embodiments, the rate of protein secretion is increased greater than 10%, 20%, 30%, 40%, 50%, 60%, 70%, 80%, 90% or 100% per hour when the rate of protein secretion is compared to the rate of protein secretion of the protein with a dsbA signal polypeptide.

In some embodiments, the invention is directed to a method of increasing the quantity of a protein secreted from a cell, comprising: (a) culturing in cell culture a host cell comprising the nucleic acid or the vector encoding the signal polypeptide as described herein, (b) inducing expression of the protein, and (c) recovering the protein secreted into the supernatant of the cell culture, wherein the quantity of protein secreted is compared to a protein with a dsbA signal polypeptide. In some embodiments, the quantity of the protein secreted from the cell is increased greater than 20% compared to a protein with a dsbA signal polypeptide. In some embodiments, the quantity of the protein secreted from the cell is increased greater than 100% compared to a protein with a dsbA signal polypeptide.

In some embodiments, the invention is directed to a method of making a protein, said method comprising: (a) culturing a host cell comprising the nucleic acid or the vector as described herein encoding the signal polypeptide of the present disclosure, so that the nucleic acid or vector is expressed, whereby upon expression of the nucleic acid or vector in the host cell, a protein encoded by the nucleic acid or vector is secreted from the cell into the supernatant; and (b) isolating the secreted protein from the supernatant. In some embodiments, the host cell or recombinant cell is Escherichia coli. In some embodiments, the host cell is cultured in cell culture under conditions in which the signal polypeptide is cleaved from the protein. In some embodiments, the methods of the invention comprise isolating the secreted protein comprises centrifugation to remove cells and/or cellular debris. In some embodiments, the methods of the invention comprise isolating the secreted protein comprises filtering to remove cells and/or cellular debris. The invention can be directed to any protein made by the methods as described herein.

In practice, heterologous proteins targeted to the periplasm are often found in the extracellular environment, possibly because of damage to or an increase in the fluidity of the outer cell membrane. The rate of this “passive” secretion may be increased by using a variety of mechanisms that permeabilize the outer cell membrane: colicin (Miksch et al. (1997) Arch. Microbiol. 167: 143-150); growth rate (Shokri et al. (2002) App Miocrobiol Biotechnol 58:386-392); TolIII overexpression (Wan and Baneyx (1998) Protein Expression Purif. 14: 13-22); bacteriocin release protein (Hsiung et al. (1989) Bio/Technology 7: 267-71), colicin A lysis protein (Lloubes et al. (1993) Biochimie 75: 451-8) mutants that leak periplasmic proteins (Furlong and Sundstrom (1989) Developments in Indus. Microbio. 30: 141-8); fusion partners (Jeong and Lee (2002) Appl. Environ. Microbio. 68: 4979-4985); recovery by osmotic shock (Taguchi et al. (1990) Biochimica Biophysica Acta 1049: 278-85). Transport of engineered proteins to the periplasmic space with subsequent localization in the broth has been used to produce properly folded and active proteins in E. coli (Wan and Baneyx, Protein Expression Purif. 14:13-22 (1998); Simmons et al., J. Immun. Meth. 263:133-147 (2002); Lundell et al., J. Indust. Microbio. 5:215-27 (1990)).

The invention can also improve recovery of “active” heterologous polypeptides/proteins. Active proteins can have a specific activity of at least about 20%, at least about 30%, at least about 40%, about 50%, about 60%, at least about 70%, about 80%, about 90%, or at least about 95% that of the native protein or polypeptide that the sequence is derived from. Further, the substrate specificity (k_{cat}/Kₘ) is optionally substantially similar to the native protein or polypeptide. Typically, k_{cat}/Kₘ will be at least about 30%, about 40%, about 50%, about 60%, about 70%, about 80%, at least about 90%, at least about 95%, or greater. Methods of assaying and quantifying measures of protein and polypeptide activity and substrate specificity (k_{cat}/Kₘ), are well known to those of skill in the art.

The activity of the heterologous polypeptide/protein can be also compared with a previously established native protein or polypeptide standard activity. Alternatively, the activity of the heterologous polypeptide/protein can be determined in a simultaneous, or substantially simultaneous, comparative assay with the native protein or polypeptide. For example, in vitro assays can be used to determine any detectable interaction between a protein or polypeptide of interest and a target, e.g. between an expressed enzyme and substrate, between expressed hormone and hormone receptor, between expressed antibody and antigen, etc. Such detection can include the measurement of colorimetric changes, proliferation changes, cell death, cell repelling, changes in radioactivity, changes in solubility, changes in molecular weight as measured by gel electrophoresis and/or gel exclusion methods, phosphorylation abilities, antibody specificity assays such as ELISA assays, etc. Generally, any in vitro or in vivo assay can be used to determine the active nature of the protein or polypeptide of interest that allows for a comparative analysis to the native protein or polypeptide so long as such activity is assayable. Alternatively, the heterologous polypeptide/protein produced in the present invention can be assayed for the ability to stimulate or inhibit interaction between the protein or polypeptide and a molecule that normally interacts with the protein or polypeptide, e.g. a substrate or a component of the signal pathway that the native protein normally interacts. Such assays can typically include the steps of combining the protein with a substrate molecule under conditions that allow the protein or polypeptide to interact with the target molecule, and detect the biochemical consequence of the interaction with the protein and the target molecule.

To measure the yield, solubility, conformation, and/or activity of the protein of interest, it may be desirable to isolate the protein from the host cell and/or extracellular medium. The isolation may be a crude, semi-crude, or pure isolation, depending on the requirements of the assay used to make the appropriate measurements. To release targeted proteins from the periplasm, treatments involving chemicals such as chloroform (Ames et al (1984) J. Bacteriol., 160: 1181-1183), guanidine-HCl, and Triton X-100 (Naglak and Wang (1990) Enzyme Microb. Technol., 12: 603-611) have been used. However, these chemicals are not inert and may have detrimental effects on many recombinant protein products or subsequent purification procedures. Glycine treatment of E. coli cells, causing permeabilization of the outer membrane, has also been reported to release the periplasmic contents (Ariga et al. (1989) J. Ferm. Bioeng., 68: 243-246). The most widely used methods of periplasmic release of recombinant protein are osmotic shock (Nosal and Heppel (1966) J. Biol. Chem., 241: 3055-3062; Neu and Heppel (1965) J. Biol. Chem., 240:3685-3692), hen eggwhite (HEW)-lysozyme/ethylenediamine tetraacetic acid (EDTA) treatment (Neu and Heppel (1964) J. Biol. Chem., 239: 3893-3900; Witholt et al. (1976) Biochim. Biophys. Acta, 443: 534-544; Pierce et al. (1995) ICheme Research. Event, 2: 995-997), and combined HEW-lysozyme/osmotic shock treatment (French et al. (1996) Enzyme and Microb. Tech., 19: 332-338). The French method involves resuspension of the cells in a fractionation buffer followed by recovery of the periplasmic fraction, where osmotic shock immediately follows lysozyme treatment.

Typically, these procedures include an initial disruption in osmotically-stabilizing medium followed by selective release in non-stabilizing medium. The composition of these media (pH, protective agent) and the disruption methods used (chloroform, HEW-lysozyme, EDTA, sonication) vary among specific procedures reported. A variation on the HEW-lysozyme/EDTA treatment using a dipolar ionic detergent in place of EDTA is discussed by Stabel et al. (1994) Veterinary Microbiol., 38: 307-314. For a general review of use of intracellular lytic enzyme systems to disrupt E. coli, see Dabora and Cooney (1990) in Advances in Biochemical Engineering/Biotechnology, Vol. 43, A. Fiechter, ed. (Springer-Verlag: Berlin), pp. 11-30.

U.S. Pat. No. 4,595,658 discloses a method for facilitating externalization of proteins transported to the periplasmic space of E. coli. This method allows selective isolation of proteins that locate in the periplasm without the need for lysozyme treatment, mechanical grinding, or osmotic shock treatment of cells. U.S. Pat. No. 4,637,980 discloses producing a bacterial product by transforming a temperature-sensitive lysogen with a DNA molecule that codes, directly or indirectly, for the product, culturing the transformant under permissive conditions to express the gene product intracellularly, and externalizing the product by raising the temperature to induce phage-encoded functions. Asami et al. (1997) J. Ferment. and Bioeng., 83: 511-516 discloses synchronized disruption of E. coli cells by T4 phage infection, and Tanji et al. (1998) 1 Ferment. and Bioeng., 85: 74-78 discloses controlled expression of lysis genes encoded in T4 phage for the gentle disruption of E. coli cells.

The proteins of this invention may be isolated and purified to substantial purity by standard techniques well known in the art, including, but not limited to, ammonium sulfate or ethanol precipitation, acid extraction, anion or cation exchange chromatography, phosphocellulose chromatography, hydrophobic interaction chromatography, affinity chromatography, nickel chromatography, hydroxylapatite chromatography, reverse phase chromatography, lectin chromatography, preparative electrophoresis, detergent solubilization, selective precipitation with such substances as column chromatography, immunopurification methods, and others. For example, proteins having established molecular adhesion properties can be reversibly fused with a ligand. With the appropriate ligand, the protein can be selectively adsorbed to a purification column and then freed from the column in a relatively pure form. The fused protein is then removed by enzymatic activity. In addition, protein can be purified using immunoaffinity columns or Ni-NTA columns. General techniques are further described in, for example, R. Scopes, Protein Purification: Principles and Practice, Springer-Verlag: N.Y. (1982); Deutscher, Guide to Protein Purification, Academic Press (1990); U.S. Pat. No. 4,511,503; S. Roe, Protein Purification Techniques: A Practical Approach (Practical Approach Series), Oxford Press (2001); D. Bollag, et al., Protein Methods, Wiley-Lisa, Inc. (1996); A K Patra et al, Protein Expr Purif, 18(2): p/182-92 (2000); and R. Mukhija, et al., Gene 165(2): p. 303-6 (1995). See also, for example, Ausubel, et al. (1987 and periodic supplements); Deutscher (1990) “Guide to Protein Purification,” Methods in Enzymology vol. 182, and other volumes in this series; Coligan, et al. (1996 and periodic Supplements) Current Protocols in Protein Science Wiley/Greene, NY; and manufacturer's literature on use of protein purification products, e.g., Pharmacia, Piscataway, N.J., or Bio-Rad, Richmond, Calif. Combination with recombinant techniques allow fusion to appropriate segments, e.g., to a FLAG sequence or an equivalent which can be fused via a protease-removable sequence. See also, for example, Hochuli (1989) Chemische Industrie 12:69-70; Hochuli (1990) “Purification of Recombinant Proteins with Metal Chelate Absorbent” in Setlow (ed.) Genetic Engineering, Principle and Methods 12:87-98, Plenum Press, NY; and Crowe, et al. (1992) QIAexpress: The High Level Expression & Protein Purification System QUIAGEN, Inc., Chatsworth, Calif.

Detection of the expressed protein is achieved by methods known in the art and include, for example, radioimmunoassays, Western blotting techniques or immunoprecipitation.

The heterologous polypeptides present in the supernatant can be separated from the host proteins by standard separation techniques well known to those of skill in the art. For example, an initial salt fractionation can separate many of the unwanted host cell proteins (or proteins derived from the cell culture media) from the heterologous polypeptide of interest. One such example can be ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

The molecular weight of the heterologous polypeptide of interest can be used to isolated it from proteins of greater and lesser size using ultrafiltration through membranes of different pore size (for example, Amicon or Millipore membranes). As a first step, the protein mixture can be ultrafiltered through a membrane with a pore size that has a lower molecular weight cut-off than the molecular weight of the protein of interest. The retentate of the ultrafiltration can then be ultrafiltered against a membrane with a molecular cut off greater than the molecular weight of the protein of interest. The heterologous polypeptide of interest will pass through the membrane into the filtrate. The filtrate can then be chromatographed as described below.

The secreted heterologous polypeptide of interest can also be separated from other proteins on the basis of its size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

In some embodiments of the present invention, more than 50% of the expressed, heterologous polypeptide or protein comprising the signal polypeptide produced can be produced in a renaturable form in a host cell. In another embodiment about 60%, 70%, 75%, 80%, 85%, 90%, 95% of the expressed protein is obtained in or can be renatured into active form.

### Example 1

In an attempt to improve the level of extracellular AT_{H35L} in the culture medium, a series of novel signal polypeptides by modification of dsbA and pelB signal polypeptides were designed. The synthesized novel signal polypeptides linked to the n-terminus of AT_{H35L} were screened in BL21Star (DE3), and extracellular AT_{H35L} in culture medium was quantified using an Octet assay and analyzed by gel-electrophoresis.

### Materials and Methods

E. coli Strains and Growth Conditions

E. coli BL21 (DE3) [fhuA2 [lon] ompT gal (λ sBamHIo ΔEcoRI-B int::(lack:PlacUv5::T7 gene1) i21 Δnin5)[dcm]ΔhsdS] and BL21 Star™ (DE3) [F⁻ ompT hsdS_{B} (n_{B}⁻, m_{B}⁻) galdcmrne131 (DE3)] were chosen to be the hosts for the expression of the constructed plasmids in this study. Recombinant cells were cultured in the seed media (20 g/L Yeast Extract) for seeding of cell and the rich growth media (20.3 g/L Yeast extract, 10.1 g/L Sodium Sulfate Anhydrous and 7 g/L K₂HPO₄) supplemented with 50 μg/mL Kanamycin for expression of recombinant AT_{H35L} proteins at 30° C.

### Construction of Expression Plasmids

pJ411 expression vector provided by DNA 2.0 Inc. (Menlo Park, Calif., USA) was used as the expression vector for T7 promoter driven-expression of signal polypeptides linked AT_{H35L} gene. Kanamycin gene was used as a selection marker in expression plasmids. Gene synthesis of all expression plasmids containing signal polypeptides linked AT_{H35L} and DNA sequencing analysis for confirmation of the synthesized plasmids were performed by DNA 2.0 Inc. Summary of signal polypeptides and recombinant plasmids for this study is listed in Table 2.

TABLE 2
Amino acid sequences of bacterial and novel signal polypeptides used.

| | Signal | SEQ | | | |
|---|---|---|---|---|---|
| Plasmid ID | Peptide ID | ID NO: | The n-region | the h-region* | the c-region^{‡} |
| DsbAss_AT_{H35L} | DsbAss | 6 | MKKI | WLALAGLVL | AFSASA |
| PedBss_AT_{H35L} | PelBss | 7 | MKYLLP | TAAAGLLLLA | AQPAMA |
| PhoAss_AT_{H35L} | PhoAss | 8 | MKQST | IALALLPLL | FTPVTKA |
| NTss_AT_{H35L} | Native ATss | 9 | MKTH | IVSSVTTTLLLGSILMN | PVANA |
| 149153 | NSP1 | 10 | MKYLLP | WLALAGLVL | AFSASA |
| 149154 | NSP2 | 11 | MKKI | TAAAGLLLLA | AFSASA |
| 149155 | NSP3 | 12 | MKKI | W¹L²A³L⁴A⁵G⁶L⁷V⁸L⁹ | AQPAMA |
| 182988 | NSP3a | 13 | MKKI | L⁹V⁸L⁷G⁶A⁵L⁴A³L²W¹ | AQPAMA |
| 182989 | NSP3b | 14 | MKKI | W¹L²A³L⁷V⁸L⁹L⁴A⁵G⁶ | AQPAMA |
| 182990 | NSP3c | 15 | MKKI | L⁴A⁵G⁶W¹L²A³L⁷V⁸L⁹ | AQPAMA |
| 182991 | NSP3d | 16 | MKKI | L⁷V⁸L⁹L⁴A⁵G⁶W¹L²A³ | AQPAMA |
| 149156 | NSP4 | 1 | MKKI | T¹A²A³A⁴G⁵L⁶L⁷L⁸L⁹A¹⁰ | AQPAMA |
| 187441 | NSP4a | 17 | MKKI | L⁶L⁷L⁸L⁹G⁵T¹A²A³A⁴A¹⁰ | AQPAMA |
| 187442 | NSP4b | 18 | MKKI | LLLLLLLLLL | AQPAMA |
| 187443 | NSP4c | 19 | MKKI | AAAAAAAAAA | AQPAMA |
| 149157 | NSP5 | 20 | MKYLLP | WLALAGLVL | AQPAMA |
| 149158 | NSP6 | 21 | MKYLLP | TAAAGLLLLA | AFSASA. |

*Numbers indicate the position of amino acids in the h-region.
^{‡}The cleavage sites are underlined in the c-region.

### Micro Fed-Batch Culture Processes

Small volume fed-batch cultures were performed in Micro 24 bioreactor (Applikon biotechnology, Forster city, CA) with 3 mL working volume. Feed medium and other supplements were sterilized by autoclaving or filtration with 0.22-μm pore size filter while the rich growth medium (20.3 g/L Yeast extract, 10.1 g/L Sodium Sulfate Anhydrous and 7 g/L K₂HPO₄) was separately autoclaved and added later under sterile conditions. The culture was inoculated with 2.8% of culture volume into the rich growth media containing 50 μg/mL of Kanamycin, 7.6 g/L of Trace metal cocktail solution (55 g/L Sodium citrate dehydrate, 27 g/L FeCl₃.6H₂O, 0.5 g/L CoCl₂.6H₂O, 0.5 g/L Na₂MoO₄.2H₂O, 0.95 g/L CuSO₄.5H₂O, 1.6 g/L MnCl₂.4H₂O, 1.3 g/L ZnCl₂ and 2 g/L CaCl₂) and 15.8 g/L of glycerol PSA. During the fed-batch cultivation, the impeller speed was initially set to 800 rpm and later controlled to keep the dissolved oxygen level (DO) at 60% saturation. In fed-batch mode of operation, 55% v/v of glycerol and 33% w/v of yeast extract solutions were used as feed media solutions. Recombinant AT_{H35L} gene expression was induced by addition of 0.5 mM isopropyl-β-D1-thiogalactopyranoside (IPTG) when cells reach at 10 of optical cell density (OD) 600, and further incubation was performed in fed-batch mode condition at 30° C. for 14 h. Cultured cells were collected at 14 h post-induction, and the supernatant of culture medium was collected from the harvest samples by centrifugation at 14,000 rpm for 5 min for analysis and quantification of extracellular AT_{H35L}. Periplasmic and cytoplasmic samples were prepared from cell lysate of the harvest samples using PeriPreps™Periplasting kit (Epicentre, Madison, Wis., USA).

### Fermentor Fed-Batch Culture Processes

Large volume fed-batch cultures were performed in a DasGip Fermentor (SaniSure Inc, Moorepark, Calif., USA) with 1 L working volume. Feed/culture media and other supplements were prepared as previously described in small volume fed-batch culture processes. The culture was inoculated with 2.8% of culture volume into the prepared culture medium containing 50 μg/ml of Kanamycin, 7.6 g/L of Trace metal cocktail solution, 15.8 g/L of glycerol PSA and 1% v/v of P2000 antifoam solution. Air space velocity was 1 vvm and the temperature was maintained at 30° C. Ammonium hydroxide (23.5% v/v) and glacial acetic acid (50% v/v) solutions were used for pH maintenance. During batch experiments, the impeller speed was initially set to 1,200 rpm and later controlled to keep the dissolved oxygen level (DO) at 60% saturation. In fed-batch mode of operation, 55% v/v of glycerol and 33% w/v of yeast extract solutions were used as feed media solutions. During feeding, the impeller speed was maintained constant at 1200 rpm, while the DO saturation was automatically kept at 60%. Recombinant AT_{H35L} gene expression was induced by addition of 0.5 mM IPTG when cells reach at 80 of OD₆₀₀, and further incubation was performed in fed-batch mode condition at 30° C. for 12 h. Cultured cells were collected at different time points post-induction to determine the profile of secreted AT_{H35L} protein productivity, osmolality and the concentration of glycerol and acetate in culture medium.

### Analyses

### SDS-PAGE Denatured Gel Electrophoresis

SDS-PAGE denatured gel electrophoresis was performed using 10% pre-cast Bis-Tris NuPAGE SDS gel (Life Technology, Frederick, Md., USA) on mini-gel tank. The running condition of electrophoresis is constant 200 V for 45 min in MOPS running buffer (Life Technology, Frederick, Md., USA). The separated protein bands were visualized by staining with the Simple blue safe stain solution (Life Technology, Frederick, Md., USA).

### Detection of AT_{H35L} Protein by Western Blot

Harvested samples were separated on SDS-PAGE under denatured condition and transferred to nitrocellulose membranes using the iblot transfer kit (Life Technology, Frederick, Md., USA). Membranes were blocked in blocking buffer (5% skim milk in 0.1% TTBS buffer) at room temperature for 1 h-1 h 30 min. Membranes were then incubated with 1 μg/mL of Mouse anti-Staphylococcal Alpha Hemolysin Toxin mAb (LC10) (MedImmune, MD, USA) in 0.1% TTBS buffer at room temperature for 1 h or at 4° C. overnight. After washing membranes with 0.1% TTBS at room temperature for 10 min in three times, incubated them with 1 μg/mL of HRP conjugated Goat anti Mouse Ab (Bethyl laboratories, Montgomery, Tex., USA) in 0.1% TTBS at room temperature for 1 h. All membranes were visualized with the SuperSignal West Pico Chemiluminescent Substrate (Pierce, Rockford, Ill., USA) and scanned on the ChemiDoc XRS™ system (Biorad, Hercules, Calif., USA).

### Quantification of Extracellular AT_{H35L} Protein

Extracellular AT_{H35L} concentration in culture medium was determined by customized Octet assay. The assay was performed on the Octet QKe (Fortebio, MenloPark, Calif., USA), and LC10 mAb (MedImmune Inc, MID, USA) was used to capture AT_{H35L} protein in culture medium. All samples were diluted by 1:10 and 1:20 ratio with the kinetics buffer (Fortebio, Menlo Park, Calif., USA) in 96-well plate (Corning, Tewksbury, Mass., USA). For the process of Octet assay, LC10 mAb was binding to Protein A biosensor (Fortebio, Menlo Park, Calif., USA) at 300 rpm for 300 sec followed by the base line step in the kinetics buffer at 300 rpm for 60 seconds, the sample association step at 300 rpm for 150 seconds, and the dissociation step in the kinetics buffer at 300 rpm for 60 seconds in the basic kinetic mode. Experimental curves were recorded for the individual sample, and data was processed and analyzed using the Octet data analysis software 7.0 (FortéBio). Finally, samples were quantified by alignment with a standard curve generated from serial dilutions of affinity-purified AT_{H35L} protein. Standard deviation values between 1:10 and 1:20 diluted samples were lower than 10%.

Purification of AT_{H35L} from Cultivation Medium

For N-terminal peptide sequence analysis, extracellular AT_{H35L} in cultivation medium was purified using a combination of ammonium sulfate precipitation and Poros XS cation exchange chromatography (Life Technologies, Grand Island, USA). Briefly, the AT_{H35L} culture was harvested by centrifugation at 850 g for 30 min to collect cultivation medium. Harvested AT_{H35L} cultivation medium was adjusted to pH 5.2 with 1 M acetic acid, and centrifuged at 9,500 g for 15 min to remove precipitant. The supernatant was then purified using ammonium sulfate precipitation followed by using Poros XS cation exchange resin for further purification.

### N-Terminal Polypeptide Sequencing

Purified AT_{H35L} was fractionated by SDS-PAGE and then transferred onto a nitrocellulose membrane using an iblot transfer kit (Life Technology, Frederick, Md., USA). The N-terminal amino acid sequencing analysis of the isolated AT_{H35L} samples were performed by Covance (Greenfield, Ind., USA) using an automated protein/peptide sequencing system.

### Results

Screening homologous and heterologous signal peptides for enhanced AT_{H35L} secretion in E. coli

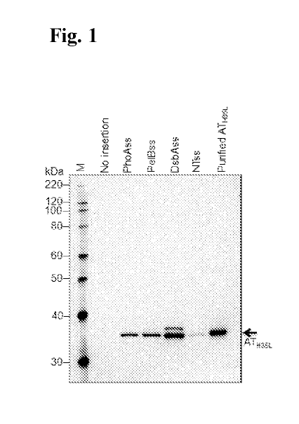

Since the selection of the signal peptide has a major impact on recombinant protein secretion in E. coli system (Sjostrom et al. 1987), various E. coli signal peptides were screened to identify a signal peptide for efficient AT_{H35L} secretion. DsbA signal sequence (dsbAss), pelB signal sequence (pelBss) and phoA signal sequence (phoAss) E. coli signal polypeptides for the Sec pathway have been selected to screen AT_{H35L} secretion. In addition, although the structural elements of signal peptides from extracellular proteins are different between S. aureus and E. coli as gram-positive bacteria and gram-negative bacteria, we also assessed the native signal peptide (NTss) of AT as some proteins are successfully secreted using their native signal peptides in a heterologous expression system (Shahhoseini et al. 2003). A summary of the signal peptides screened is shown in Table 2. Recombinant cell strains containing the different signal peptide-AT_{H35L} fusion constructs were evaluated in fed-batch culture using a micro24 bioreactor system. Signal polypeptide linked AT_{H35L} is processed to yield a secretory protein of approximately 33 kDa in E. coli expression system. Since signal polypeptide consists of 20-26 amino acids, mature AT_{H35L} and AT_{H35L} preprotein can be separated on a denaturing gel by size differences. Extracellularly secreted AT_{H35L} in culture medium was confirmed and relatively quantified using Western blot (FIG. 1). Western blot analysis confirmed that AT_{H35L} is secreted into the extracellular surroundings (culture medium) (FIG. 1) as well as the periplasmic space (data not shown) after the export of AT_{H35L} from the cytoplasmic space. Also, a relative quantification of extracellular AT_{H35L} by Western blot showed that dsbAss showed the highest efficiency of extracellular AT_{H35L} product among other signal polypeptides, while NTss showed the lowest efficiency of extracellular AT_{H35L} product. Amino acid sequences similarity between E. coli signal polypeptides and NTss is at very low (data not shown) and this observation implies that the native signal polypeptide of AT may not efficiently lead the translocation of AT_{H35L} in E. coli. PelBss linked AT_{H35L} showed lower extracellular AT_{H35L} productivities compared with dsbAss (FIG. 1). The data indicate that various E. coli signal polypeptides affect the secretion efficiency of AT_{H35L} differently, although dsbAss, pelBss and phoAss have high similarity of amino acid sequences and used the SEC pathway for secretion of recombinant proteins in E. coli (Natale et al., Biochimica et Biophysica Acta 1778:1735-1756 (2008)). In particular, the identity of amino acid sequence between dsbAss and pelBss is 45%. The present data suggests that the selection of a signal polypeptide is critical for secretion of AT_{H35L} in E. coli although the signal polypeptides have very high similarity of amino acid sequences alignment. In addition, AT_{H35L} secretion could not efficiently proceed by its native signal polypeptide in E. coli unlike in S. Aureus.

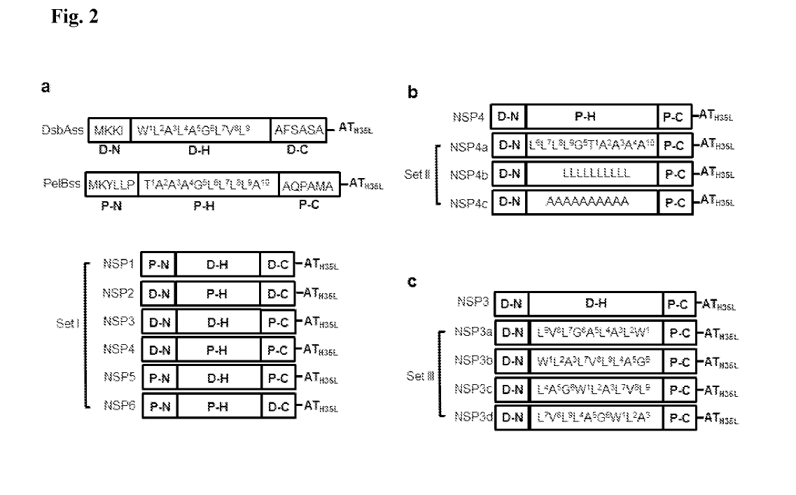

Screening the selected bacterial signal polypeptides on AT_{H35L} secretion showed that the secretion of AT_{H35L} has different impact in between dsbAss and pelBss (FIG. 1) although they display high similarity (45% identity) of the amino acid sequences. The result suggested that individual signal polypeptides influence the secretion process of AT_{H35L}, and it may be related to the function of each region in signal polypeptide. Most signal polypeptides share some common structural features; they consist of an N-terminal polar region (the n-region) followed by a hydrophobic core region (the h-region), and a polar cleavage region (the c-region). The basic n-region is 2-5 amino acids long, and has a net positive charge. The h-region normally contains between 6 and 15 amino acid residues, and has been found to be the most essential part of the signal polypeptide for translocation and targeting to the transmembrane (Sjostrom et al. 1987; Choi et al. 2004). The data suggests secretion efficiency could be influenced by structural changes of signal polypeptide through swiping regions between two different signal polypeptides. The secretion efficiency of AT_{H35L} was investigated by structurally swiping domains of two signal polypeptides, which results in improving the yield of extracellular AT_{H35L}. Novel signal polypeptides for this study and the schematic diagram of novel signal polypeptide constructs are represented in Table 2 and FIG. 2, respectively.

### Improving the Yield of Extracellular AT_{H35L} by Novel Signal Polypeptides

Set I novel signal polypeptides were created by swiping the n-region, the h-region and the c-region of dsbAss and pelBss (Table 2 and FIG. 2A). With regards to set I novel signal polypeptides, six novel signal polypeptides were created by swiping each region of dsbAss and pelBss (NSP1 thru NSP6), and NSP1 through NSP6 and linking the resulting signal polypeptide to AT_{H35L} (plasmids 149153 thru 149158) (FIG. 2). Recombinant plasmids containing novel signal polypeptide linked AT_{H35L} were cultured in 1L scale fed-batch conditions for screening.

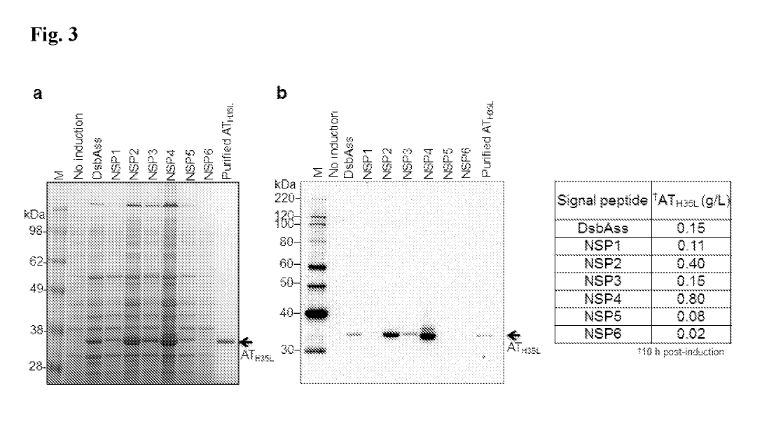

Recombinant AT_{H35L} gene expression was induced by addition of 0.5 mM IPTG when the cell density reached at OD₆₀₀ of 80, followed by incubation at 30° C. in fed-batch mode for a further 12 h. The value of OD₆₀₀ at the end of the fed-batch process reached 120-140 with viability ≥90% (data not shown). For analysis of extracellular AT_{H35L}, the supernatant of culture medium was collected from the harvested cultivation samples by centrifugation at 10 h post-induction. The concentration of extracellular AT_{H35L} was determined by an Octet assay and the size of processed mature AT_{H35L} in culture medium was verified by SDS-PAGE (FIG. 3). The data show that NSP2 and NSP4 increased the yield of secreted AT_{H35L} in the culture medium by 2.5-fold (0.4 g/L) and 5-fold (0.8 g/L) respectively compared with dsbAss (0.15 g/L). Intriguingly, these two novel signal peptides share the n-region of dsbAss and the h-region of pelBss in their structures with only the c-region being different (Table 1). The c-regions of NSP2 and NSP4 had not been mutated by either addition or truncation of amino acids, so the signal peptidase (SP) cleavage site was not modified and therefore did not affect the cleavage process by SP. The observed improvement in secretion suggests that the amino acids following the hydrophobic region may influence the structural formation of the h-region in the signal peptide and the translocation of AT_{H35L} across the cytoplasmic membrane.

In addition, the productivity of secreted AT_{H35L} was reduced in NSP6 (0.02 g/L) compared with dsbAss (0.15 g/L). NSP2 and NSP6 share the same h- and the c-region domains in their structures except the n-region domain. The present data denote that the n-region of dsbAss is a favorable region in translocation of AT_{H35L} to easily access to the cytoplasm membrane (FIG. 3). Also, NSP3 could not improve the yield of secretory AT_{H35L} (0.15 g/L) while NSP4 made the best productivity of secretory of AT_{H35L} (0.8 g/L) although they share the n-region domain of dsbAss and the c-region domain of pelBss in their signal polypeptide structures except the h-region.

Based on the result, the data suggest that amino acids following after the h-region significantly influence the conformation of signal polypeptide structure to make AT_{H35L} feasible to access the cytoplasmic membrane. Also, conjugated domain of the n-region of dsbAss and the h-region of pelBss is a favorable structure domain to translocation of AT_{H35L} in the secretion process.

Altering the Position of Amino Acids in the h-Region Domain

The result of set I novel signal polypeptide screening showed that there are different effects on the AT_{H35L} secretion efficiency between NSP3 and NSP4, which are having different hydrophobic regions (FIG. 3). Since the h-region plays an important role in translocation of recombinant proteins to the cytoplasmic membrane (Hikita et al., J Biol Chem 267:4882-4888 (1992); Chou et al., J Biol Chem 265:2873-2880 (1990); MacFarlane et al., Eur J Biochem 233:766-771 (1995); Phoenix et al., J Biol Chem 268:17069-18073 (1993); Fekkes et al., Microbiol and Molec Biol Review 63:161-173 (1999)), a couple of sets of novel signal polypeptides were created by modifying the position of amino acids in the h-regions of NSP3 and NSP4, and investigated whether modification of the amino acid position in the h-region affects the secretion efficiency of AT_{H35L} protein. In NSP3 and NSP4 structures, the h-region domain of NSP3 consists of leucine and valine which are strong hydrophobic amino acids, while the h-region domain of NSP4 mainly consists of polyleucine and polyalanine. In generation of NSP3 and NSP4 mutants except NSP4b and NSP4c, total hydrophobicity and the residue length in the h-region have not changed since only the amino acid position was altered.

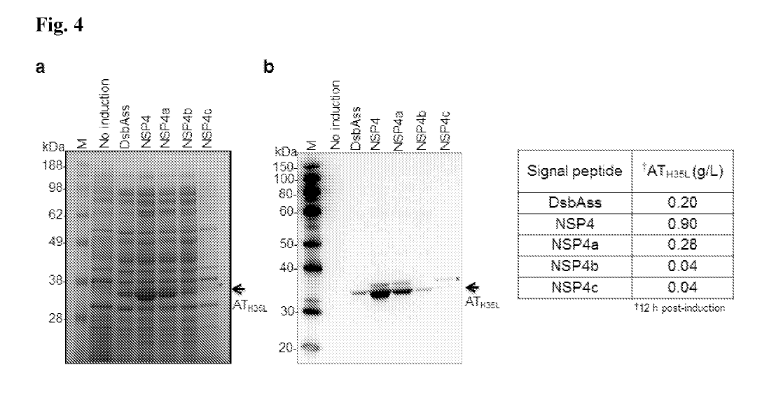

With regards to engineering of NSP4, three mutants were created to change a hydrophobicity of the h-region (set II signal polypeptides): i) the position of a polyleucine stretch and the position of a polyalanine stretch in the h-region (NSP4a) were swiped, ii) all residues in the h-region were replaced with polyleucine residues to create a stretch of strong hydrophobic amino acids (NSP4b), iii) all residues in the h-region were replaced with polyalanine residues to create a stretch of weak hydrophobic amino acids (NSP4c) (FIG. 2b). FIG. 4 indicates that NSP4 mutant signal polypeptides reduced overall AT_{H35L} secretion levels. NSP4 produced 0.9 g/L of secretory AT_{H35L} in culture medium but the yield of secretory AT_{H35L} was reduced by 0.28 g/L when NSP4a was conjugated to AT_{H35L}. NSP4a has alternative positions of polyleucine and polyalanine in the h-region domain from NSP4. It notes that there is no total hydrophobicity or the residues length change in the h-region. The changed position of hydrophobic amino acids in the h-region domain significantly affected the translocation of AT_{H35L} protein. Also, the yield of secretory AT_{H35L} in both NSP4b AT_{H35L} and NSP4c AT_{H35L} was significantly reduced by 0.04 g/L. Although the h-region of NSP4b consists of only strong hydrophobic amino acid by polyleucine, the strong hydrophobic region showed less efficiency in translocation of AT_{H35L} in the secretion process (0.04 g/L). Not surprisingly, there is no detection of secretory AT_{H35L} in NSP4c by Western blot since the h-region domain of NSP4c is composed with only polyalanine which is weaker hydrophobic amino acid. Although 0.04 g/L of AT_{H35L} was detected by the quantification assay, this amount of AT_{H35L} represent AT_{H35L} precursor released from the intracellular space due to cell death. During the fed-batch cultivation, NSP4 mutants altered the characteristic of cell growth. In particular, cell growth rate of NSP4c AT_{H35L} was lower than other mutant constructs. The cell growth rate of NSP4c AT_{H35L} at OD₆₀₀ was reduced after 10 h post-induction due to initiation of cell apoptosis (data not shown). Moreover, secretory AT_{H35L} in NSP4c at either 12 h post-induction or earlier induction time points was not detected. Cell apoptosis in cultivation of NSP4c AT_{H35L} occurred quickly during the expression of AT_{H35L} after induction, which results in reduction of the secretory AT_{H35L} in culture medium.

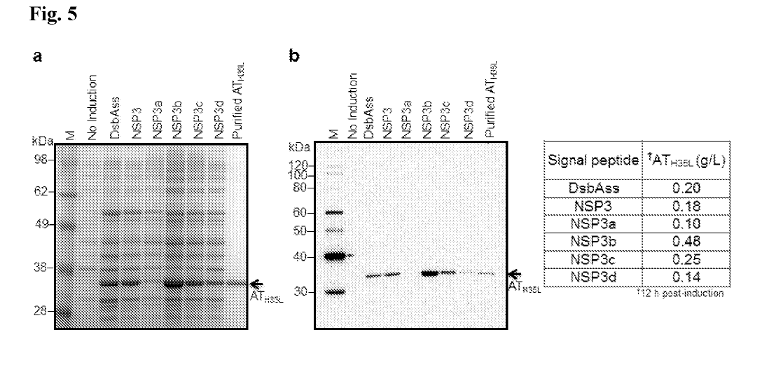

Based on the result of set I signal polypeptide screening, NSP3 could not improve the secretion of AT_{H35L}, even if it contains the same n- and c-region domains of NSP4 (FIGS. 2a and 3). Also, the yield of secretory AT_{H35L} has been markedly changed when NSP4a is linked to AT_{H35L} which has the altered position of polyleucine and polyalanine residues (FIGS. 2b and 4). Thus, the data indicates that a hydrophobicity plot in the h-region has an important factor in translocation of AT_{H35L}. Four mutants of NSP3 were created by changing the position of amino acid residues in the h-region domain to make various hydrophobicity plots of the h-region (FIG. 2c). The position change of amino acid in the h-region domain significantly impacts the secretion efficiency of AT_{H35L} (FIG. 5). NSP3b improved the yield of secretory AT_{H35L} by 2 fold (0.48 g/L) compared with NSP3 (0.18 g/L). In contrast, NSP3a and NSP3d reduced the yield of secretory AT_{H35L} by 0.1 g/L and 0.14 g/L, respectively (FIG. 5). In the h-region of NSP3b, strong hydrophobic amino acid residues such as leucine and valine are located in the center of the h-region by changing the position of residues. In other NSP3 mutant signal polypeptides, the position of strong hydrophobic residues is located close to the n- or c-regions, which is differing from NSP3b. Previous several studies have been shown that total hydrophobicity and hydrophobic domain length are important factors for recombinant proteins translocation and the substrate specificity in recognizing by a factor(s) involved in the secretory machinery (Bankaitis et. al, Cell 37:243-252 (1984); Emr et al., Nature 285:82-85 (1980); Hikita et al., J Biol Chem 267:4882-4888 (1992)). However, the results suggested that the location of hydrophobic amino acid residues also an important factor for protein translocation. In spite of no change of total hydrophobicity and the length of the hydrophobic domain, alternative positions of amino acid residues in the h-region domain made an effect on AT_{H35L} translocation. In the characteristic of cell growth, cell growth rate of all mutant constructs was various during the fed-batch culture process. The present work suggests that a hydrophobicity plot of the h-region domain significantly impacts the translocation process of AT_{H35L} protein and cell growth during expression and secretion of AT_{H35L} in E. coli. (FIGS. 4 and 5). Also, total hydrophobicity of the h-region is indispensable in secretion of AT_{H35L} protein (FIG. 4) as shown in previous studies (Emr et al. 1980; Hikita et al. 1992).

Further characterization of NSP4 on the secretion of AT_{H35L} This study demonstrates that NSP4 improved the yield of secretory AT_{H35L} in culture medium by ≥5 fold compared with dsbAss at 10 h post-induction (FIG. 3). During the fed-batch cultivation, it was shown that the characteristic of cell containing NSP4 AT_{H35L} has been changed after induction. To optimize the induction length of time for NSP4 AT_{H35L}, we compared the yield of secretory AT_{H35L} between NSP4 and dsbAss at various induction time points, and verified AT_{H35L} precursor contamination by gel-electrophoresis.

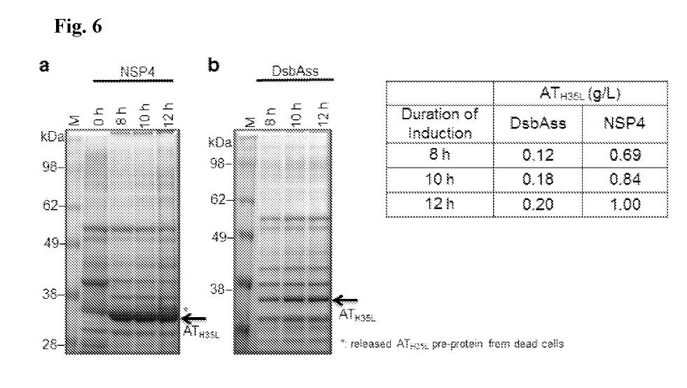

Growth of fed-batch cultures carried out in 1L fermentor at 30° C., pH 7 and with 0.5 mM IPTG for AT_{H35L} gene induction. Details of the fed-batch culture conditions for recombinant AT_{H35L} gene expression are described in this study. Cultivation samples of both dsbAss_AT_{H35L} and NSP4_AT_{H35L} were collected every 2 h after induction, and culture medium containing secretory AT_{H35L} was separated from the cultivation samples by centrifugation for quantification of secretory AT_{H35L}. Verification of secretory AT_{H35L} and AT_{H35L} precursor was performed by gel-electrophoresis and Western blot (FIG. 6a, b). NSP4_AT_{H35L} and dsbAss_AT_{H35L} produced 0.7 g/L and 0.12 g/L of secretory AT_{H35L} respectively when samples were harvested at 8 h post-induction. The yield of secretory AT_{H35L} was increased up to 1 g/L with NSP4 and 0.2 g/L with dsbAss at 12 h post-induction (FIG. 6a, b). Quantification data showed that the yield of secretory AT_{H35L} in culture medium was increased up to 12 h post-induction in both dsbAss_AT_{H35L} and NSP4_AT_{H35L}. However, analytical data by electrophoresis revealed that AT_{H35L} precursor of NSP4_AT_{H35L} was leaked out from the intracellular space into culture medium from 12 h post-induction due to cell apoptosis. Cell viability of dsbAss_AT_{H35L} was ≥90% at 12 h post-induction, while cell viability of NSP4_AT_{H35L} initiated to drop to 90% (data not shown). The cell viability result was correlated to gel-electrophoresis analysis (FIG. 6a, b). These data suggest that NSP4 is very efficient on the translocation of AT_{H35L}, and increased the productivity of secretory AT_{H35L} by 6 fold compared with dsbAss at 6 h post-induction (FIG. 6). Moreover, NSP4 released mature AT_{H35L} in shorten induction time. Decreasing of culture process time for recombinant proteins production would be very beneficial in commercial activities for saving a process time.

In the secretion process of recombinant proteins, the cleavage of signal polypeptide by signal peptidase is an important and useful criterion of protein translocation across the membrane to release a mature protein properly into the periplasmic space and the extracellular surroundings. Commonly, E. coli signal polypeptide has the Ala-X-Ala specific motif (the A-X-A domain) as the cleavage recognition site in the c-region, and all of novel signal polypeptides in this study contain the A-X-A domain in their c-region. To verify the cleavage of AT_{H35L} from signal polypeptide, secretory AT_{H35L} in culture medium was purified as described in this study and the purified AT_{H35L} protein was confirmed the secretion of mature AT_{H35L} by proper splicing process using N-terminal peptide sequencing analysis. We confirmed that novel signal polypeptides in this study were cleaved properly from AT_{H35L} precursor without interruption of the cleavage process using the N-terminal peptide sequencing analysis (Table 3).

TABLE 3
N-terminal peptide sequencing analysis of
secreted AT_{H35L} in culture medium.
Identified peptide sequences

| The position of | Released AT_{H35L} | |
|---|---|---|
| amino acid | from NSP4 | Mature AT_{H35L} |
| 1 | A | A |
| 2 | D | D |
| 3 | S | S |
| 4 | D | D |
| 5 | I | I |
| 6 | N | N |
| 7 | I | I |
| 8 | K | K |
| 9 | T | T |

### Discussion

Signal polypeptide linked AT_{H35L} is expressed and secreted as a soluble protein in E. coli expression system (Menzies et al., Infect Immun 64:1839-1841 (1994)). In the present study, we screened a native signal polypeptide of AT (NTss), DsbAss, pelBss and phoAss on AT_{H35L} protein secretion into culture medium. DsbAss, pelBss and phoAss E. coli signal polypeptides are commonly used for secretion of recombinant proteins in the SEC system of E. coli. In screening, signal polypeptides linked AT_{H35L} is extracellularly secreted from the periplasmic space into culture medium. Although some soluble AT_{H35L} is still left as a soluble protein in the cytoplasmic space (data not shown), 30% of expressed AT_{H35L} protein was secreted into the periplasmic space and culture medium. In particular, dsbAss-linked AT_{H35L} has the best secretion efficiency among the selected signal polypeptides, and pelBss linked AT_{H35L} showed the lowest secretion efficiency of AT_{H35L} among E. coli signal polypeptides (FIG. 2). Although there is a high similarity of amino acid sequence among E. coli signal polypeptides, they showed different efficiencies on secretion of AT_{H35L} proteins due to various functions of each region in signal polypeptide during the secretion process (Hikita et al. (1992), Ismail et al., Biotech Lett 33:999-1005 (2011), Velaithan et al., Ann Microbiol 64:543-550 (2014)). Intriguingly, amino acid sequence alignments between pelBss and dsbAss showed at high similarity with 45% identity, but pelBss linked AT_{H35L} was inefficiently secreted secretory AT_{H35L} into culture medium unlike dsbAss (FIG. 2).

Improvement of extracellular AT_{H35L} secretion product using new novel signal polypeptides was investigated, specifically looking the effects of individual signal polypeptide region on secretion of extracellular AT_{H35L}. During the investigation, it was found that newly created novel signal polypeptides on AT_{H35L} secretion, NSP1, NSP5 and NSP6 reduced the yield of secretory AT_{H35L} at 10 h post-induction compared with dsbAss, while NSP2 and NSP4 increased the yield of secretory AT_{H35L} by 3 fold and 5 fold, respectively (FIG. 3). In the structure of set I signal polypeptides, the big difference among these signal polypeptides is that NSP2 and NSP4 contain the n-region of dsbAss, while NSP1, NSP5 and NSP6 contain the n-region of pelBss. The result implies the n-region of dsbAss has better efficiency on recognition of the n-region by nascent signal polypeptide recognition particle (SRP) than the n-region of pelBss after translation of AT_{H35L} protein. A number of studies have been shown that signal polypeptide was mutated by point-mutagenesis for increasing total hydrophobicity of the h-region or changing a stretch length of signal polypeptide to improve the secretion of recombinant proteins (Chou et al., J Biol Chem 265:2873-2880 (1990), Hikita et al. 1992, Jonet et al. 2012, Klatt et al., Microbial. Cell Factories 11:97-106 (2012)). This study investigated novel signal polypeptides created by cross-transition of two different E. coli signal polypeptides having high similarity, and these novel signal polypeptides contributed in improving the yield of secretory AT_{H35L}.

In the secretion pathway, many components are involved in the secretion process. At the initiation of secretion process, the n-region with basic amino acids of signal polypeptide was recognized by SRP to access the cytoplasmic membrane. The NSP4 structure is composed with the n-region of dsbAss which contains more basic amino acid residues such as polylysine compared to the n-region of pelBss. It makes NSP4 more easily accessible to the cytoplasmic membrane. The h-region is also an important factor to translocate recombinant proteins, and this study demonstrated that the secretion efficiency of recombinant proteins was changed depending on the location of hydrophobic amino acid residues in the h-region regardless total hydrophobicity. Both NSP2 and NSP4 increased the yield of secretory AT_{H35L} and these two signal polypeptides contain the n-region of dsbAss and the h-region of pelBss. However, NSP4 produced better amount of secretory AT_{H35L} than NSP2 in the same amount of cell. Both signal polypeptides have the Ala-X-Ala specific site as the cleavage recognition site for the splicing process. Nonetheless, the h- and c-regions of pelBss showed better efficiency in translocation and releasing of AT_{H35L} than that of dsbAss. The data suggests that the c-region of pelBss may be more efficient in the cleavage process than that of dsbAss since there is no difference of total AT_{H35L} protein expression between two signal polypeptides. Alternatively, some of amino acids in the c-region may affect to the structure conformation of h-region domain, which results in influence the translocation efficiency of AT_{H35L}.

Intriguingly, NSP3 linked AT_{H35L} did not significantly improve the secretion of AT_{H35L} although NSP3 contains the same n- and c-regions of NSP4 except the h-region (FIGS. 2 and 3). Thus, the data indicates the h-region of pelBss plays a critical role in translocation and secretion of AT_{H35L}. Total hydrophobicity of the h-region domain is an important factor when a recombinant protein access to the cytoplasmic membrane for translocation. For this reason, the h-region of signal polypeptide has been studied by addition or truncation of amino acids to change the total hydrophobicity for improvement of recombinant protein secretion in E. coli. (Jonet et al. (2012); Low et al., Bioengineered 3:334-338 (2011); Phoenix et al. 1993; Sjostrom et al. 1987; Velaithan et al. 2014). In the current study, mutants of NSP3 and NSP4 were created by changing the amino acid position in the h-region of these two signal polypeptides since these two signal polypeptides have different hydrophobic regions but the same n- and c-regions (FIGS. 2b and c).

In general, the h-region consists of weak and strong hydrophobic amino acid residues. Thus, how the h-region with only strong hydrophobic residues affects AT_{H35L} secretion (NSP4b) was investigated. For the comparison of NSP4b, another mutant was created with only polyalanine to disrupt hydrophobicity in signal polypeptide structure (NSP4c). Interestingly, the secretion efficiency of AT_{H35L} has been significantly reduced with NSP4b and NSP4c as well (FIG. 4). Many of studies have been shown that increasing of hydrophobicity in signal polypeptides enhanced the secretion efficiency of recombinant proteins (Bankaitis et al., Cell 37:243-252 (1984); Emr et al. (1980); Hikita et al. (1992)). However, the composition of only strong hydrophobic residues could not enhance the translocation of AT_{H35L}. The present data suggest that the h-region requires a proper combination of strong and weak hydrophobic amino acid residues for the balance of hydrophobicity.

Although total hydrophobicity or a stretch of the h-region has not been changed in NSP3 and NSP4 mutants, the efficiency of AT_{H35L} secretion showed noticeable differences among these mutants by changing of the amino acid position (FIGS. 4 and 5). The position of amino acids in the h-region significantly affects the translocation of recombinant AT_{H35L} protein in the secretion process. The present study is a new finding that the secretion efficiency of recombinant proteins can be improved by changing the amino acid position in the h-region domain.

Signal polypeptide mutants also affect the characteristic of cell growth after induction of AT_{H35L} gene expression. During the fed-batch cultivation, the data suggests the optimized culture process time for NSP4 was 10 h after induction for the control of AT_{H35L} product quality. In contrast, the optimized culture process time for dsbAss was 12 h after induction to achieve the maximum secretory AT_{H35L} product. A 5 fold higher yield of secretory AT_{H35L} from NSP4 than dsbAss was achieved (FIG. 6). The data indicates that NSP4 has major influence on improvement of AT_{H35L} secretion in shorten culture processing time, suggesting that this signal polypeptide is very useful for commercial levels of secretory AT_{H35L} products in E. coli expression system.

In summary, the data show that some of the synthetic signal polypeptides improved the secretion efficiency of AT_{H35L} compared with non-modified signal polypeptides. In particular, NSP4 improved the yield of secreted AT_{H35L} by 4-fold in the fed-batch fermentation process. In this work, the present findings indicate that the secretion efficiency of AT_{H35L} was significantly improved by modification of the h-region of signal polypeptides and the position of residues in the h-region also influences the secretion efficiency. These new novel signal polypeptides can be used to improve the secretion efficiency of heterologous proteins in E. coli.

In conclusion, the present data here demonstrate that i) the n-region of dsbAss including additional basic amino acid residues was a favorable domain compared to the n-region of pelBss, ii) a hydrophobicity in the h-region is critical for translocation of recombinant proteins to the cytoplasmic membrane, iii) the h-region requires both weak and strong amino acid residues properly for efficient translocation of recombinant proteins, iv) shuffling of hydrophobic amino acid residues in the h-region significantly affect translocation of recombinant proteins, v) in particular, NSP4 significantly influenced on improvement of AT_{H35L} secretion in shorten culture processes. We believe that designed novel signal peptides in the present study could be used to improve the secretion efficiency of a recombinant protein in the E. coli expression platform.

Unless defined otherwise, all technical and scientific terms and any acronyms used herein have the same meanings as commonly understood by one of ordinary skill in the art in the field of this invention. Any polypeptide or nucleic acid sequence, protein, vector, compositions, cell, methods and means for communicating information similar or equivalent to those described herein can be used to practice this invention.

All references cited herein are incorporated herein by reference to the full extent allowed by law. The discussion of those references is intended merely to summarize the assertions made by their authors. No admission is made that any reference (or a portion of any reference) is relevant prior art. Applicants reserve the right to challenge the accuracy and pertinence of any cited reference.

What is claimed is:

1. A signal polypeptide comprising an amino acid sequence having at least 90% sequence identity to any one of SEQ ID NOS: 1 or 10 to 21.

2. The signal polypeptide of claim 1, comprising an amino acid sequence having at least 95% sequence identity to any one of SEQ ID NOS: 1 or 10 to 21.

3. The signal polypeptide of claim 1, comprising an amino acid sequence of any one of SEQ ID NOS: 1 or 10 to 21.

4. A protein comprising (i) a signal polypeptide comprising an amino acid sequence having at least 90% sequence identity to any one of SEQ ID NOS: 1 or 10 to 21, and (ii) a heterologous polypeptide.

5. The protein of claim 4, wherein the protein comprises (i) a signal polypeptide comprising an amino acid sequence having at least 95% sequence identity to any one of SEQ ID NOS: 1 or 10 to 21, and (ii) a heterologous polypeptide.

6. The protein of claim 4, wherein the protein comprises (i) a signal polypeptide comprising an amino acid sequence to any one of SEQ ID NOS: 1 or 10 to 21, and (ii) a heterologous polypeptide.

7. The protein of any one of claims 4 to 6, wherein the heterologous polypeptide comprises greater than 20 amino acids.

8. The protein of any one of claims 4 to 7, wherein the heterologous polypeptide has a molecular weight of 25 kDa to 50 kDa.

9. The protein of any one of claims 4 to 8, wherein the heterologous polypeptide has a molecular weight of 30 kDa to 35 kDa.

10. The protein of any one of claims 4 to 9, wherein the heterologous polypeptide is selected from the group consisting of an enzyme, toxin, antibody, antibody fragment, antigen, therapeutic protein, and combination thereof.

11. A signal polypeptide comprising an amino acid sequence having at 90% sequence identity to SEQ ID NO: 1.

12. The signal polypeptide of claim 11, comprising an amino acid sequence having at least 95% sequence identity to SEQ ID NO: 1.

13. The signal polypeptide of claim 11, comprising an amino acid sequence of SEQ ID NO: 1.

14. A protein comprising (i) a signal polypeptide comprising an amino acid sequence having at least 90% sequence identity to SEQ ID NO: 1, and (ii) a heterologous polypeptide.

15. The protein of claim 14, wherein the protein comprises (i) a signal polypeptide comprising an amino acid sequence having at least 95% sequence identity to SEQ ID NO: 1, and (ii) a heterologous polypeptide.

16. The protein of claim 14, wherein the protein comprises (i) a signal polypeptide comprising an amino acid sequence of SEQ ID NO: 1, and (ii) a heterologous polypeptide.

17. The protein of any one of claims 14 to 16, wherein the heterologous polypeptide comprises greater than 20 amino acids.

18. The protein of any one of claims 14 to 17, wherein the heterologous polypeptide has a molecular weight of 25 kDa to 50 kDa.

19. The protein of any one of claims 14 to 18, wherein the heterologous polypeptide has a molecular weight of 30 kDa to 35 kDa.

20. The protein of any one of claims 14 to 19, wherein the heterologous polypeptide is selected from the group consisting of an enzyme, toxin, antibody, antibody fragment, antigen, therapeutic protein, and combination thereof.

21. The protein of any one of claims 14 to 20, wherein the heterologous polypeptide comprises Alpha Toxin (AT).

22. The protein of any one of claims 14 to 21, wherein the heterologous polypeptide comprises Alpha Toxin from Staphylococcus aureus.

23. The protein of any one of claims 21 to 22, wherein the Alpha Toxin comprises a substitution at the amino acid position corresponding to H35.

24. The protein of claim 23, wherein the substitution is a H35L substitution.

25. A protein comprising the amino acid sequence of SEQ ID NO: 2.

26. A composition comprising a signal polypeptide according to any one of claims 1 to 3 or 11 to 13, or protein according to claims 4 to 10 or 14 to 25.

27. A nucleic acid encoding the signal polypeptide of any one of claims 1 to 3 or 11 to 13, or protein according to claims 4 to 10 or 14 to 25.

28. A nucleic acid (1) encoding the signal polypeptide of any one of claims 1 to 3 or 11 to 13, and (2) comprising one or more restriction enzyme sites.

29. A vector comprising the nucleic acid of claim 27 or claim 28.

30. The vector of claim 29, further comprising an origin of replication.

31. The vector of claim 29 or 30, further comprising a promoter sequence operably linked to the nucleic acid.

32. The vector of any one of claims 29 to 31, wherein the promoter sequence is operable in a prokaryote.

33. The vector of any one of claims 29 to 32, wherein the vector is a plasmid, a transposon, or a viral vector.

34. A recombinant cell engineered to express the protein of any one of claims 4 to 10 or 14 to 25.

35. The recombinant cell of claim 34 which is a prokaryote cell.

36. The recombinant cell of any one of claims 34 to 35, wherein the prokaryote cell is of the genus Escherichia.

37. The recombinant cell of any one of claims 34 to 36, wherein the prokaryote cell is Escherichia coli.

38. A host cell transformed with the vector of any one of claims 29 to 33.

39. The host cell of claim 38, wherein the cell is a prokaryote cell.

40. The host cell of claim 38 or claim 39, wherein the prokaryote cell is of the genus Escherichia.

41. The host cell of any one of claims 38 to 40, wherein the prokaryote cell is Escherichia coli.

42. A method of producing the protein of any one of claims 4 to 10 or 14 to 25, comprising culturing a recombinant cell engineered to express the protein, or a host cell transformed with a vector encoding the protein, under conditions in which the protein is expressed.

43. The method of claim 42, wherein the recombinant cell or host cell is cultured in cell culture under conditions in which the protein is secreted from the recombinant cell or host cell.

44. The method of claim 42 or claim 43, wherein the recombinant cell or host cell is cultured in cell culture under conditions in which the signal polypeptide is cleaved from the protein.

45. The method of any one of claims 42 to 44, further comprising recovering the protein from the cell culture.

46. The method of claim 45, wherein recovering the protein comprises centrifugation to remove cells and/or cellular debris.

47. The method of claim 45 or claim 46, wherein recovering the protein comprises filtering to remove cells and/or cellular debris.

48. A method of increasing the rate of protein secretion from a cell, comprising: (a) culturing in cell culture a host cell comprising the nucleic acid of claim 27 or the vector of any one of claims 29 to 33 which encodes the protein, (b) inducing expression of the protein, and (c) recovering the protein secreted into the supernatant of the cell culture, wherein the rate of protein secretion is compared to the rate of protein secretion of the protein with a dsbA signal polypeptide.

49. The method of claim 48, wherein the recovering of (c) occurs between 8 and 12 hours after the inducing of (b).

50. The method of claim 48 or claim 49, wherein the rate of protein secretion is increased greater than 20% per hour.

51. A method of increasing the quantity of a protein secreted from a cell, comprising: (a) culturing in cell culture a host cell comprising the nucleic acid of claim 27 or the vector of any one of claims 29 to 33 which encodes the protein, (b) inducing expression of the protein, and (c) recovering the protein secreted into the supernatant of the cell culture, wherein the quantity of protein secreted is compared to a protein with a dsbA signal polypeptide.

52. The method of claim 51, wherein the quantity of the protein secreted from the cell is increased greater than 20% compared to a protein with a dsbA signal polypeptide.

53. The method of claim 51, wherein the quantity of the protein secreted from the cell is increased greater than 100% compared to a protein with a dsbA signal polypeptide.

54. A method of making a protein, said method comprising
a. culturing a host cell comprising the nucleic acid of claim 27 or the vector of any one of claims 29 to 33, so that the nucleic acid is expressed, whereby upon expression of the nucleic acid or vector in the host cell, a protein encoded by the nucleic acid or vector is secreted from the cell into the supernatant; and
b. isolating the secreted protein from the supernatant.

55. The method of any one of claims 41 to 54, wherein the host cell or recombinant cell is Escherichia coli.

56. The method of claim 54 or claim 55, wherein the host cell is cultured in cell culture under conditions in which the signal polypeptide is cleaved from the protein.

57. The method of any one of claims 54 to 56, wherein isolating the secreted protein comprises centrifugation to remove cells and/or cellular debris.

58. The method of any one of claims 54 to 56, wherein isolating the secreted protein comprises filtering to remove cells and/or cellular debris.

59. A protein made by the method of any one of claims 42 to 58.


SEQUENCE LISTING (30 sequences)

SEQ ID NO: 1 - protein, 20 aa, Artificial Sequence
  Description of Artificial Sequence Synthetic peptide
MKKITAAAGLLLLAAQPAMA

SEQ ID NO: 2 - protein, 313 aa, Artificial Sequence
  Description of Artificial Sequence Synthetic polypeptide
MKKITAAAGLLLLAAQPAMAADSDINIKTGTTDIGSNTTVKTGDLVTYDKENGMLKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 3 - protein, 293 aa, Staphylococcus aureus
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 4 - protein, 293 aa, Staphylococcus aureus
ADSDINIKTGTTDIGSNTTVKTGDLVTYDKENGMLKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 5 - DNA, 882 bp, Artificial Sequence
  Description of Artificial Sequence Synthetic polynucleotide
gcagacagcgacatcaacattaagactggtaccaccgacatcggcagcaatacgaccgttaaaaccggcgacctggtgacctacgataaagagaatggcatgttgaaaaaagttttctactcttttatcgatgataagaatcacaacaaaaagctgctggtcattcgtacgaagggcaccatcgcgggtcagtatcgcgtctactccgaagagggcgcgaacaagagcggtctggcttggccgagcgcatttaaggtccagctgcaactgcctgataacgaagttgcgcagattagcgactactacccacgcaatagcattgacaccaaagagtatatgagcaccctgacgtatggcttcaatggtaacgtgaccggcgacgacacgggtaagatcggtggtctgatcggcgccaatgtgagcatcggtcatacgctgaaatatgttcagccggacttcaagacgattttggagtccccgacggacaaaaaagttggctggaaagtgattttcaacaacatggtcaatcaaaattggggtccgtacgatcgtgacagctggaacccggtgtatggtaatcaactgtttatgaaaacccgcaacggttctatgaaagcggccgacaacttcctggatccgaataaggctagctccctgctgtcgagcggttttagcccggattttgcaacggtgattaccatggaccgtaaggcgagcaagcaacagaccaatatcgacgtcatttacgaacgtgttcgtgatgattatcagctgcactggactagcaccaactggaagggtaccaacaccaaggataaatggattgatcgctcaagcgaacgttacaagatcgattgggagaaagaagagatgacgaactaa

SEQ ID NO: 6 - protein, 19 aa, Artificial Sequence
  Description of Artificial Sequence Synthetic peptide
MKKIWLALAGLVLAFSASA

SEQ ID NO: 7 - protein, 22 aa, Artificial Sequence
  Description of Artificial Sequence Synthetic peptide
MKYLLPTAAAGLLLLAAQPAMA

SEQ ID NO: 8 - protein, 21 aa, Artificial Sequence
  Description of Artificial Sequence Synthetic peptide
MKQSTIALALLPLLFTPVTKA

SEQ ID NO: 9 - protein, 26 aa, Artificial Sequence
  Description of Artificial Sequence Synthetic peptide
MKTHIVSSVTTTLLLGSILMNPVANA

SEQ ID NO: 10 - protein, 21 aa, Artificial Sequence
  Description of Artificial Sequence Synthetic peptide
MKYLLPWLALAGLVLAFSASA

SEQ ID NO: 11 - protein, 20 aa, Artificial Sequence
  Description of Artificial Sequence Synthetic peptide
MKKITAAAGLLLLAAFSASA

SEQ ID NO: 12 - protein, 19 aa, Artificial Sequence
  Description of Artificial Sequence Synthetic peptide
MKKIWLALAGLVLAQPAMA

SEQ ID NO: 13 - protein, 19 aa, Artificial Sequence
  Description of Artificial Sequence Synthetic peptide
MKKILVLGALALWAQPAMA

SEQ ID NO: 14 - protein, 19 aa, Artificial Sequence
  Description of Artificial Sequence Synthetic peptide
MKKIWLALVLLAGAQPAMA

SEQ ID NO: 15 - protein, 19 aa, Artificial Sequence
  Description of Artificial Sequence Synthetic peptide
MKKILAGWLALVLAQPAMA

SEQ ID NO: 16 - protein, 19 aa, Artificial Sequence
  Description of Artificial Sequence Synthetic peptide
MKKILVLLAGWLAAQPAMA

SEQ ID NO: 17 - protein, 20 aa, Artificial Sequence
  Description of Artificial Sequence Synthetic peptide
MKKILLLLGTAAAAAQPAMA

SEQ ID NO: 18 - protein, 20 aa, Artificial Sequence
  Description of Artificial Sequence Synthetic peptide
MKKILLLLLLLLLLAQPAMA

SEQ ID NO: 19 - protein, 20 aa, Artificial Sequence
  Description of Artificial Sequence Synthetic peptide
MKKIAAAAAAAAAAAQPAMA

SEQ ID NO: 20 - protein, 21 aa, Artificial Sequence
  Description of Artificial Sequence Synthetic peptide
MKYLLPWLALAGLVLAQPAMA

SEQ ID NO: 21 - protein, 22 aa, Artificial Sequence
  Description of Artificial Sequence Synthetic peptide
MKYLLPTAAAGLLLLAAFSASA

SEQ ID NO: 22 - protein, 6 aa, Artificial Sequence
  Description of Artificial Sequence Synthetic 6xHis tag
HHHHHH

SEQ ID NO: 23 - protein, 10 aa, Artificial Sequence
  Description of Artificial Sequence Synthetic peptide
LLLLGTAAAA

SEQ ID NO: 24 - protein, 10 aa, Artificial Sequence
  Description of Artificial Sequence Synthetic peptide
LLLLLLLLLL

SEQ ID NO: 25 - protein, 10 aa, Artificial Sequence
  Description of Artificial Sequence Synthetic peptide
AAAAAAAAAA

SEQ ID NO: 26 - protein, 9 aa, Artificial Sequence
  Description of Artificial Sequence Synthetic peptide
LVLGALALW

SEQ ID NO: 27 - protein, 9 aa, Artificial Sequence
  Description of Artificial Sequence Synthetic peptide
WLALVLLAG

SEQ ID NO: 28 - protein, 9 aa, Artificial Sequence
  Description of Artificial Sequence Synthetic peptide
LAGWLALVL

SEQ ID NO: 29 - protein, 9 aa, Artificial Sequence
  Description of Artificial Sequence Synthetic peptide
LVLLAGWLA

SEQ ID NO: 30 - protein, 9 aa, Staphylococcus aureus
ADSDINIKT